(19) United States
(12) Patent Application Publication
(10) Pub. No.: US 2025/0017888 A1
(43) Pub. Date: Jan. 16, 2025

(54) COMPOUNDS FOR TREATING DISEASES ASSOCIATED WITH HYALURONAN OVERPRODUCTION

(71) Applicant: THE ROYAL VETERINARY COLLEGE, London (GB)

(72) Inventors: Peter PREHM, Hamburg (DE); Behzad JAVAHERI, London (GB); Andrew PITSILLIDES, London (GB)

(73) Assignee: THE ROYAL VETERINARY COLLEGE, London (GB)

(21) Appl. No.: 18/264,774

(22) PCT Filed: Feb. 16, 2022
(86) PCT No.: PCT/GB2022/050421

§ 371 Date: Aug. 9, 2023

(30) Foreign Application Priority Data
Feb. 17, 2021 (GB) 2102243.9

(51) Int. Cl. A61K 31/24

(52) U.S. Cl. CPC A61K 31/24

(57) ABSTRACT

There are provided compounds of formula (I): [hyaluronan production inhibitor]-[labile linker]-X (I), which compounds are useful in the treatment of diseases associated with hyaluronan overproduction.

### FIELD OF THE INVENTION

The field of the present invention relates to compounds, compositions comprising such compounds, and the use of such compounds and compositions in medicine.

The present invention relates to novel compounds, compositions comprising such compounds, and the use of such compounds and compositions in medicine. In particular, the present invention relates to the use of such compounds and compositions in methods for the treatment and prevention of diseases associated with hyaluronan overproduction, such as rheumatoid diseases (e.g. arthritis and osteoarthritis), which treatment and prevention is thought to occur through inhibition of hyaluronan production.

### BACKGROUND OF THE INVENTION

The listing or discussion of an apparently prior-published document in this specification should not necessarily be taken as an acknowledgement that the document is part of the state of the art or is common general knowledge.

Hyaluronan is synthesised by alternate transfer of the precursor nucleotide sugars UDP-GlcNac and UDP-GIcA to the reducing end of UDP-hyaluronan (Prehm, P., Biochem. J., 1983, 211, 181-189; Prehm, P. Biochem. J., 1983, 211, 191-198). In contrast to other glycosaminoglycans, hyaluronan is synthesised at the plasma membrane and directly exported into the extracellular matrix (Prehm, P., Biochem. J., 1990, 267, 185-189). It is exported from fibroblasts and chondrocytes by the ABC transporter MRP5 (Prehm, P. and Schumacher, U., Biochem, Pharmacol., 2004, 68, 1401-1410; Schulz, T. et al., J Biol. Chem., 2007, 282, 20999-21004) and from epithelial cells by CFTR (Schulz, T. et al., Pathobiology, 2010, 77, 200-209).

Hyaluronan overproduction is associated with a variety of diseases and disorders, including myocardial infarction, stroke, metastasis of tumours and rheumatoid diseases (for serum hyaluronan as a disease marker, see e.g.: Laurent T C, Laurent U B, Fraser J R, Ann Med., 1996, 28:241-53; for liver fibrosis, see e.g.: Yang Y M et al, Sci Transl Med. 2019, 11 (496):eaat9284; for cancer, see e.g.: Itano N, Kimata K. Semin Cancer Biol. 2008. 18(4):268-74; for inflammatory bowel disease, see e.g: Kessler S P, Obery D R, de la Motte C. Int J Cell Bio. 2015; 2015:745237; for osteoarthritis, see e.g: Stracke D, Schulz T, Prehm P. Mol Nutr Food Res. 2011, 55(3):485-94; for heart infarct, see e.g.: Petz A et al, Circ Res. 2019. 124(10):1433-1447; for kidney dysfunction, see e.g.: Hansell P, Palm F. Acta Physiol (Oxf). 2015. 213(4):795-804; for autoimmune diseases, see e.g: Nagy N et al Matrix Biol. 2019, 78-79:292-313; for rheumatoid arthritis, see e.g: Yoshioka Y et al Arthritis Rheum. 2013. 65(5):1160-70; for rheumatoid diseases, see e.g. Hochberg, M. C et al Rheumatology 2018. Elsevier; Pitsillides A A et al, Arthritis Rheum. 1994. 37(7):1030-8).

Rheumatoid diseases affect many tissues including joints tendons, ligaments, bones, and muscles.

Common symptoms include joint pain, loss of motion in a joint or joints, inflammation-swelling, redness, and warmth in a joint or affected area. Most of these conditions are a consequence of an inappropriate immunological reaction to body tissues. In the case of rheumatoid arthritis the immune system reacts to joint tissues.

Osteoarthritis and rheumatoid arthritis are both forms of arthritis (disease of the joint).

Unlike most other rheumatoid diseases, osteoarthritis does not appear to be primarily linked to problems with the immune system. Instead, osteoarthritis appears to result from trauma and damage to the joint and cartilage. The diagnostic criteria of osteoarthritis are changes visible on an X-ray of the joint. The X-ray may show narrowing of the joint space or the presence of bone spurs. Definitive diagnosis is usually provided by MRI (magnetic resonance imaging) of the joint (Chaudhari A S et al, J Magn Reson imaging. 2020. 52(5):1321-1339).

Osteoarthritis is characterised by erosion of cartilage, proteolysis of aggrecan and collagen, and disturbed synthesis rates of aggrecan and hyaluronan by chondrocytes. Hyaluronan overproduction is a recognised early change in osteoarthritis pathogenesis.

Key events in osteoarthritic cartilage include increased hyaluronan production, decreased aggrecan synthesis and proteolytic cleavage of collagen type 11 and aggrecan core protein.

In healthy cartilage, the synthesis of hyaluronan and proteoglycans are finely tuned to each other and most of the hyaluronan is endocytosed after binding to intact CD44. Osteoarthritic cartilage shows profound (15-fold) increases in hyaluronan production that accompany reduced proteoglycan production (60%) and greater shedding from cartilage into the environment (D'Souza, A. L. et al, Arch. Biochem. Biophys. 2000, 374, 59-65). The main reason for this shedding is proteolytic cleavage of CD44. Hyaluronan overproduction also contributes to the enlargement of hypertrophic lacunae in the growth plate and reduced aggrecan binding to hyaluronan induces apoptosis.

Hyaluronan overproduction is a very early reaction of tissue activation and its purpose is to provide the swelling of the tissue due its enormous hydration volume; this response ‘opens’ the tissue for ingression by immune cells for repair. Hyaluronan overproduction thus occurs in most tissues as a prelude to inflammation. However, cartilage is a special tissue in that it is so densely packed, with profound amounts of extracellular matrix, that lymphocytes are unable to enter the damaged site for repair. The traumatised cartilage tissue nonetheless still reacts with hyaluronan overproduction resulting in irreversible destruction. The increase in hyaluronan overproduction at the very early stage of osteoarthritis development is indeed established in the literature.

Although increased hyaluronan production has been known for a long time to precede loss of proteoglycan and collagen degradation, until recently it had been challenging to interfere with this process because of the lack of available hyaluronan production inhibitors.

The only inhibitor of hyaluronan production which has been investigated in detail is 4-methylumbelliferone. Its mode of action is indirect, as it depletes the intracellular UDP-GIcA pool of precursors for hyaluronan synthesis. For this reason, side effects must be expected on the glucuronidation reaction of biotransformation or on the production of glucuronic acid-containing proteoglycans, such as those with chondroitin sulfate glysosaminoglycans (Urakawa, H. et al, Int. J. Cancer. 2012, 130, 454-466; Piccioni, F. et al, Glycobiology., 2012, 22, 400-410; Nagy, N. et al, Circulation, 2010, 122, 2313-2322; Twarock, S. J. Biol. Chem., 2010, 285, 23276-23284).

It has been reported that hyaluronan is exported by the ATP-binding MRP5 (Stracke D. et al, Mol. Nutr. Food Res., 2011, 485-494). Inhibitors of MRP5 have been described (Stracke D. et al, Mol. Nutr. Food Res., 2011, 485-494; Prehm, P., Food. Chem. Toxicol., 2013, 76-81).

To the best of our knowledge, there has been no effective therapy of a disease associated with hyaluronan overproduction to date, wherein the mechanism of action of the therapeutic involves inhibition of hyaluronan production.

Moreover, in the case of osteoarthritis, despite its prevalence, to date there is no effective therapy that alters the course of the disease or repairs existing damage. Currently, the therapeutic strategy in the treatment of osteoarthritis is purely symptomatic, consisting of anti-inflammatory drugs, analgesics, physiotherapy and massages.

Given this, at present there remains a need for alternative, effective therapies for diseases associated with hyaluronan overproduction, and in particular, there remains a need for an effective therapy for osteoarthritis.

Madelmont et al have explored the possibility of attaching quaternary ammonium groups to various molecules (Madelmont et al., Bioorg. Med. Chem. 2007, 15, 2368-2374; Madelmont et al., Bioorg. Med. Chem. 2003, 11, 5007-5012; Madelmont et al., Bioconjug. Chem. 2003, 14, 500-506; Madelmont et al, Bioconjug. Chem. 2000, 11, 212-218; Madelmont et al, J. Med. Chem. 1999, 42, 5235-5240; Vidal et al, Eur. J. Med. Chen. 2010, 45, 405-410; US 2004208822).

The present invention is focused on both inhibiting the early increases in hyaluronan synthesis and effectively targeting the cartilage in order to limit any potential serious, systemic off-target effects, by using the compounds disclosed below.

### DETAILED DESCRIPTION OF THE INVENTION

It has now been surprisingly found that hyaluronan production inhibitor compounds, tethered through a labile linker to a cationic moiety (or a masked cationic moiety) are able to effectively inhibit the production of hyaluronan, and have properties rendering them useful for the treatment and prevention of diseases characterised by hyaluronan overproduction, such as rheumatoid diseases, in particular arthritis and osteoarthritis.

Without being bound by theory, it is believed that the compounds of the invention act as prodrugs. The cationic moiety is believed to target hyaluronan production sites, such as those near the cartilage. The labile ester linker is thought to undergo hydrolysis, such as by plasma esterases (for instance, non-specific esterases in synovial fluid of osteoarthritic joints that are particularly focussed at the site of erosion). Therefore, it is expected that the hyaluronan production inhibitor is preferentially liberated from the linker at the desired site, near a hyaluronan production site, such as at the eroded cartilage.

Compounds of the invention containing a masked charged moiety (e.g. masked thiazolium) are thought to be converted to the charged analogues (e.g. to the thiazolium salt) in vivo. In turn, the resulting charged compound is thought to act as a prodrug, as outlined above. The compounds containing the masked thiazolium are believed to be particularly suitable for oral administration due to their increased lipophilicity.

Compounds of the invention containing a long chain fatty acid counterion are believed to be particularly suitable for topical administration due to superior transdermal delivery.

### Compounds of the Invention

The invention relates to compounds of formula (I):

[hyaluronan production inhibitor]-[labile linker]-X (1)
or a pharmaceutically-acceptable salt thereof,
wherein the hyaluronan production inhibitor fragment is selected from the group consisting of Formula A, B, C, D, and E, wherein
formula A is:

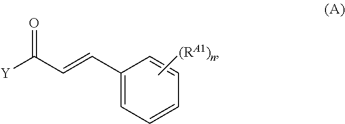

wherein Y is a group selected from the group consisting of:

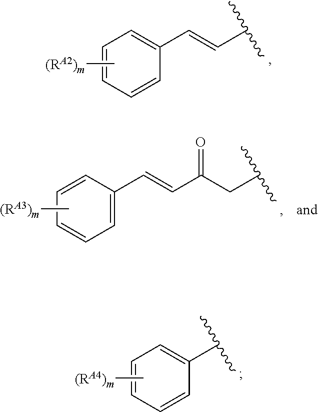

R^{A1}, R^{A2}, R^{A3}, and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2}, R^{A3} or R^{A4} group is

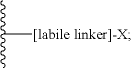

n is 0 to 5;
m is 0 to 5; and
R^{A5} is C₁₋₆ alkyl, C₂₋₆ alkenyl, C₂₋₆ alkynyl, which three groups are optionally substituted by one or more groups selected from halo, —OH and —OMe;
formula B is:

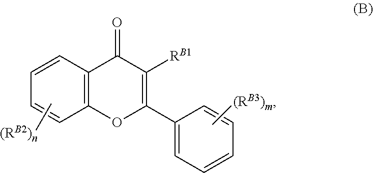

wherein R^{B1}, R^{B2}, and R^{B3} are each independently selected from the group consisting of —OH, —ORB⁴, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe; and one R^{B2} or R^{N3} group is

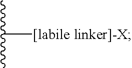

n is 0 to 4;
m is 0 to 5;
R^{B4} is C₁₋₆ alkyl, C₂₋₆ alkenyl, C₂₋₆ alkynyl, which three groups are optionally substituted by one or more groups selected from halo, —OH and —OMe;
formula C is:

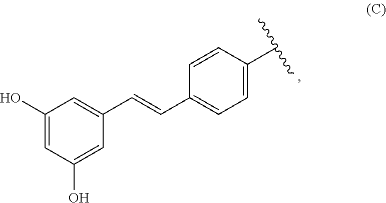 [SMILES: *c1ccc(/C=C/c2cc(O)cc(O)c2)cc1]

wherein the wavy line represents the point of attachment to the labile linker fragment; formula D is:

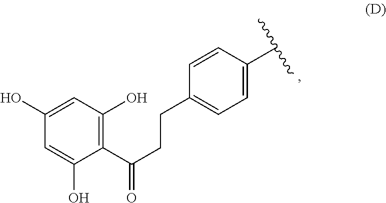 [SMILES: *c1ccc(CCC(=O)c2c(O)cc(O)cc2O)cc1]

wherein the wavy line represents the point of attachment to the labile linker fragment; or formula E is:

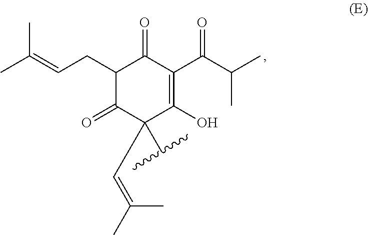 [SMILES: *C1(C=C(C)C)C(=O)C(CC=C(C)C)C(=O)C(C(=O)C(C)C)=C1O]

wherein the wavy line represents the point of attachment to the labile linker fragment;
the labile linker fragment is —OC(O)R^{L1}—;
wherein R^{L1} is selected from C₁₋₆ alkylene, C₂₋₆ alkenylene, C₂₋₆ alkynylene, arylene, or heteroarylene, which five groups are optionally substituted with one or more groups independently selected from halo, —OR^{L2} or ═O;
R^{L2} represents H or C₁₋₆ alkyl;
X is selected from the group consisting of quaternary ammonium, quaternary phosphonium, pyridinium and thiazolium salts; or
X is

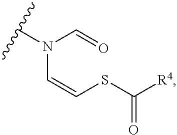

wherein R⁴ is C₁₋₆ alkyl.

Compounds of formula I, including particularly compounds of formula Ia, as defined hereinbelow (including pharmaceutically acceptable salts thereof) may be referred to herein as the “compounds of the invention”.

In a first aspect of the invention, there is provided a compound of formula Ia

[hyaluronan production inhibitor]-[labile linker]-X (Ia)
or a pharmaceutically-acceptable salt thereof,
wherein the hyaluronan production inhibitor fragment is selected from the group consisting of Formula A, B, C, D, and E, wherein
formula A is:

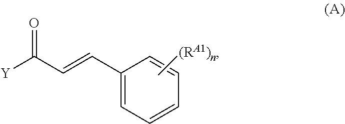

wherein Y is a group selected from the group consisting of:

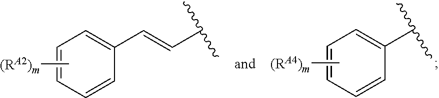

R^{A1}, R^{A2}, and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2} or R^{A4} group is

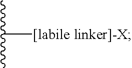

n is 0 to 5;
m is 0 to 5; and
R^{A5} is C₁₋₆ alkyl, C₂₋₆ alkenyl, C₂₋₆ alkynyl, which three groups are optionally substituted by one or more groups selected from halo, —OH and —OMe;
formula B is:

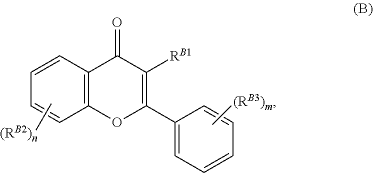

wherein R^{B1}, R^{B2}, and R^{B3} are each independently selected from the group consisting of —OH, —OR^{B4}, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe; and one R^{B2} or R^{B3} group is

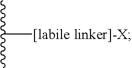

n is 0 to 4;
m is 0 to 5;
R^{B4} is C₁₋₆ alkyl, C₂₋₆ alkenyl, C₂₋₆ alkynyl, which three groups are optionally substituted by one or more groups selected from halo, —OH and —OMe;
formula C is:

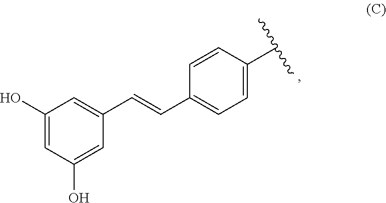 [SMILES: *c1ccc(/C=C/c2cc(O)cc(O)c2)cc1]

wherein the wavy line represents the point of attachment to the labile linker fragment;
formula D is:

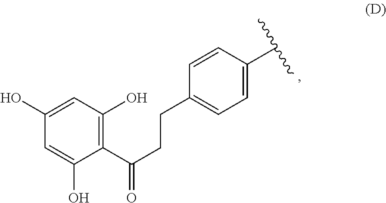 [SMILES: *c1ccc(CCC(=O)c2c(O)cc(O)cc2O)cc1]

wherein the wavy line represents the point of attachment to the labile linker fragment; or
formula E is:

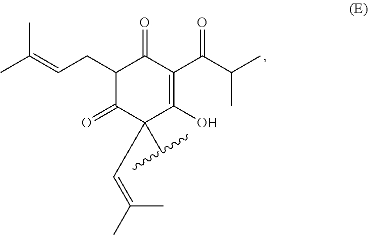 [SMILES: *C1(C=C(C)C)C(=O)C(CC=C(C)C)C(=O)C(C(=O)C(C)C)=C1O]

wherein the wavy line represents the point of attachment to the labile linker fragment; the labile linker fragment is —OC(O)R^{L1}—;
wherein R^{L1} is selected from C₁₋₆ alkylene, C₂₋₆ alkenylene, C₂₋₆ alkynylene, arylene, or heteroarylene, which five groups are optionally substituted with one or more groups independently selected from halo, —OR^{L2} or ═O;
R^{L2} represents H or C₁₋₆ alkyl;
X is selected from the group consisting of quaternary ammonium, quaternary phosphonium, pyridinium and thiazolium salts; or
X is

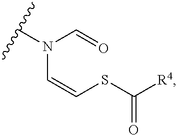

wherein R⁴ is C₁₋₆ alkyl.

For the avoidance of doubt, the skilled person will understand that references herein to compounds of particular aspects of the invention (such as the first aspect of the invention, i.e. referring to compounds of formula Ia as defined in the first aspect of the invention) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular acid addition salts that may be mentioned include those formed by reaction with corresponding acids, thus protonating the compound of the invention, to form carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed by reaction with corresponding bases, thus removing a proton from compounds of the invention, to form salts with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds of the invention may exist as solids, and thus the scope of the invention includes all amorphous, crystalline and part crystalline forms thereof, and may also exist as oils. Where compounds of the invention exist in crystalline and part crystalline forms, such forms may include solvates, which are included in the scope of the invention.

For the avoidance of doubt, compounds of the invention may also exist in solution (i.e. in solution in a suitable solvent). For example, compounds of the invention may exist in aqueous solution, in which case compounds of the invention may exist in the form of hydrates thereof.

Compounds of the invention may contain double bonds and, unless otherwise indicated, may thus exist as E (entgegen) and Z (zusammen) geometric isomers about each individual double bond. Unless otherwise specified, all such isomers and mixtures thereof are included within the scope of the invention.

Compounds of the invention may also exhibit tautomerism. All tautormeric forms and mixtures thereof are included within the scope of the invention (particularly those of sufficient stability to allow for isolation thereof).

Compounds of the invention may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

For the avoidance of doubt, the skilled person will understand that where a particular group is depicted herein as being bound to a ring system via a floating bond (i.e. a bond not shown as being bound to a particular atom within the ring), the relevant group may be bound to any suitable atom within the relevant ring system (i.e. the ring within which the floating bond terminates).

Unless otherwise specified, C_{E}z alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z} cycloalkyl group). When there is a sufficient number (i.e. a minimum of four) of carbon atoms, such groups may also be part cyclic (so forming a C_{4-z} partial cycloalkyl group). For example, cycloalkyl groups that may be mentioned include cyclopropyl, cyclopentyl and cyclohexyl. Similarly, part cyclic alkyl groups (which may also be referred to as “part cycloalkyl” groups) that may be mentioned include cyclopropylmethyl. When there is a sufficient number of carbon atoms, such groups may also be multicyclic (e.g. bicyclic or tricyclic) and/or spirocyclic. For the avoidance of doubt, particular alkyl groups that may be mentioned include straight chain (i.e. not branched and/or cyclic) alkyl groups.

Unless otherwise specified, C_{2-z} alkenyl groups (where z is the upper limit of the range) defined herein may be straight-chain or, when there is a sufficient number (i.e. a minimum of three) of carbon atoms, be branched-chain, and/or cyclic (so forming a C_{4-z} cycloalkenyl group). When there is a sufficient number (i.e. a minimum of five) of carbon atoms, such groups may also be part cyclic. For example, part cyclic alkenyl groups (which may also be referred to as “part cycloalkenyl” groups) that may be mentioned include cyclopentenylmethyl and cyclohexenylmethyl. When there is a sufficient number of carbon atoms, such groups may also be multicyclic (e.g. bicyclic or tricyclic) or spirocyclic. For the avoidance of doubt, particular alkenyl groups that may be mentioned include straight chain (i.e. not branched and/or cyclic) alkenyl groups.

Unless otherwise specified, C_{2-z} alkynyl groups (where z is the upper limit of the range) defined herein may be straight-chain or, when there is a sufficient number (i.e. a minimum of four) of carbon atoms, be branched-chain. For the avoidance of doubt, particular alkynyl groups that may be mentioned include straight chain (i.e. not branched and/or cyclic) alkynyl groups.

For the avoidance of doubt, C_{2-z} alkenyl and C_{2-z} alkynyl groups, when there is a sufficient number of carbon atoms, may have one or more carbon-carbon double and/or triple bonds. For the avoidance of doubt, unless otherwise specified, groups referred to herein as “alkyl”, “alkenyl” and/or “alkynyl” will be taken as referring to the highest degree of unsaturation in a bond present in such groups. For example, such a group having a carbon-carbon double bond and, in the same group, a carbon-carbon triple bond will be referred to as “alkynyl”. Alternatively, it may be particularly specified that such groups will comprise only the degree of unsaturation specified (i.e. in one or more bond therein, as appropriate; e.g. in one bond therein).

For the avoidance of doubt, alkyl, alkenyl and alkynyl groups as described herein may also act as linker groups (i.e. groups joining two or more parts of the compound as described), in which case such groups may be referred to as “alkylene”, “alkenylene” and/or “alkynylene” groups, respectively.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulfur (e.g. oxygen, nitrogen and sulfur, such as oxygen and nitrogen).

As used herein, the term heterocyclyl may refer to non-aromatic monocyclic and polycyclic (e.g. bicyclic) heterocyclic groups (which groups may, where containing a sufficient number of atoms, also be bridged) in which at least one (e.g. one to four) of the atoms in the ring system is other than carbon (i.e. a heteroatom), and in which the total number of atoms in the ring system is between three and twelve (e.g. between five and ten, such as between three and eight; for example, forming a 5- or 6-membered heterocyclyl group). Further, such heterocyclyl groups may be saturated, forming a heterocycloalkyl, or unsaturated containing one or more carbon-carbon or, where possible, carbon-heteroatom or heteroatom-heteroatom double and/or triple bonds, forming for, example a C_{2-z} (e.g. C_{4-z}) heterocycloalkenyl (where z is the upper limit of the range) or a C_{7-z}, heterocycloalkynyl group.

For the avoidance of doubt, the skilled person will understand that heterocyclyl groups that may form part of compounds of the invention are those that are chemically obtainable, as known to those skilled in the art. Various heterocyclyl groups will be well-known to those skilled in the art, such as 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridinyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuryl, tetrahydropyridinyl (such as 1,2,3,4-tetrahydropyridinyl and 1,2,3,6-tetrahydropyridinyl), thietanyl, thiiranyl, thiolanyl, tetrahydrothiopyranyl, thiomorpholinyl, trithianyl (including 1,3,5-trithianyl), tropanyl and the like.

Substituents on heterocyclyl groups may, where appropriate, be located on any atom in the ring system including a heteroatom. Further, in the case where the substituent is another cyclic compound, then the cyclic compound may be attached through a single atom on the heterocyclyl group, forming a spirocyclic compound. The point of attachment of heterocyclyl groups may be via any suitable atom in the ring system, including (where appropriate) a further heteroatom (such as a nitrogen atom), or an atom on any fused carbocyclic ring that may be present as part of the ring system. Heterocyclyl groups may also be in the N- or S-oxidised forms, as known to those skilled in the art.

At each occurrence when mentioned herein, particular heterocyclyl groups that may be mentioned include 3- to 8-membered heterocyclyl groups (e.g. a 4- to 6-membered heterocyclyl group, such as a 5- or 6-membered heterocyclyl group).

For the avoidance of doubt, references to polycyclic (e.g. bicyclic or tricyclic) groups (for example when employed in the context of heterocyclyl or cycloalkyl groups (e.g. heterocyclyl)) will refer to ring systems wherein at least two scissions would be required to convert such rings into a non-cyclic (i.e. straight or branched) chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, to groups in which two non-adjacent atoms are linked by an alkyl (which, when linking two moieties, may be referred to as alkylene) group (optionally containing one or more heteroatoms), which later groups may be referred to as bridged, or to groups in which the second ring is attached to a single atom, which latter groups may be referred to as spiro compounds.

Particular heterocyclyl groups that may be mentioned include piperidinyl (e.g. piperidin-1-yl), octahydro-1H-isoindolyl (e.g. octahydro-1H-isoindol-2-yl), azetidinyl (e.g. azetidine-1-yl), oxetanyl (e.g. oxetan-3-yl), morpholinyl (e.g. morpholin-4-yl), piperazinyl (e.g. piperazin-1yl or piperazin-4-yl), azepanyl (e.g. azepan-1-yl), imidazolidinyl (e.g. imidazolidine-2-yl), pyrrolidinyl (e.g. pyrrolidine-1yl), and diazepanyl (e.g. 1,4-diazepan-1-yl).

As may be used herein, the term aryl may refer to C₆₋₁₄ (e.g. C₆₋₁₀) aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl, and the like). For the avoidance of doubt, the point of attachment of substituents on aryl groups may be via any suitable carbon atom of the ring system.

For the avoidance of doubt, the skilled person will understand that aryl groups that may form part of compounds of the invention are those that are chemically obtainable, as known to those skilled in the art. Particular aryl groups that may be mentioned include phenyl and naphthyl, such as phenyl.

As may be used herein, references to heteroaryl (with may also be referred to as heteroaromatic) groups may refer to 5- to 14-(e.g. 5- to 10-) membered heteroaromatic groups containing one or more heteroatoms (such as one or more heteroatoms selected from oxygen, nitrogen and/or sulfur). Such heteroaryl groups may comprise one, two, or three rings, of which at least one is aromatic. Substituents on heteroaryl/heteroaromatic groups may, where appropriate, be located on any suitable atom in the ring system, including a heteroatom (e.g. on a suitable N atom).

The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocyclyl ring.

For the avoidance of doubt, the skilled person will understand that heteroaryl groups that may form part of compounds of the invention are those that are chemically obtainable, as known to those skilled in the art. Various heteroaryl groups will be well-known to those skilled in the art, such as pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, pyrazolopyrimidinyl, indazolyl, pyrimidinyl, quinolinyl, isoquinolinyl, quinazolinyl, benzofuranyl, benzothiophenyl, benzoirnidazolyl, benzoxazolyl, benzothiazolyl, benzotriazolyl and purinyl.

For the avoidance of doubt, the oxides of heteroaryl/heteroaromatic groups are also embraced within the scope of the invention (e.g. the N-oxide).

As stated above, heteroaryl includes polycyclic (e.g. bicyclic) groups in which one ring is aromatic (and the other may or may not be aromatic). Hence, other heteroaryl groups that may be mentioned include groups such as benzo[1,3]dioxolyl, benzo[1,4]dioxinyl, dihydrobenzo[d]isothiazole, 3,4-dihydrobenz[1,4]oxazinyl, dihydrobenzothiophenyl, indolinyl, 5H,6H,7H-pyrrolo[1,2-b]pyrimidinyl, 1,2,3,4-tetrahydroquinolinyl, thiochromanyl and the like.

Particular heteroaryl groups that may be mentioned include morpholine, piperidine, pyridine, pyrazine, pyridazine, pyrazole.

For the avoidance of doubt, where a ring is depicted having circle therein, its presence shall indicate that the relevant ring is aromatic. Alternatively, aromatic groups may be depicted as cyclic groups comprising therein a suitable number of double bonds to allow for aromaticity.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature).

All isotopes of any particular atom or element as specified herein are contemplated within the scope of the compounds of the invention. Hence, the compounds of the invention also include deuterated compounds, i.e. compounds of the invention in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more R^{A1} groups are present, those R^{A1} groups may be the same or different. Similarly, where two or more R^{A1} groups are present and each represent C₁₋₆ alkyl, the C₁₋₆ alkyl groups in question may be the same or different.

Also for the avoidance of doubt, when a term such as “0 to 5” is employed herein, this will be understood by the skilled person to mean 0 and 5, inclusively. Unless otherwise stated, the same reasoning will apply to other such terms used herein.

Further for the avoidance of doubt, when it is specified that a substituent is itself optionally substituted by one or more substituents (e.g. C₁₋₆ alkyl optionally substituted by one or more groups selected from halo, OH and —OMe), these substituents where possible may be positioned on the same or different atoms. Such optional substituents may be present in any suitable number thereof (e.g. the relevant group may be substituted with one or more such substituents, such as one such substituent).

For the avoidance of doubt, where groups are referred to herein as being optionally substituted it is specifically contemplated that such optional substituents may be not present (i.e. references to such optional substituents may be removed), in which case the optionally substituted group may be referred to as being unsubstituted.

The symbol [symbol] , displayed perpendicular to a bond, indicates the point at which the displayed moiety is attached to the remainder of the molecule.

For the avoidance of doubt, the skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

In particular embodiments (i.e. particular embodiments of the first aspect of the invention), the compound of formula Ia is such that the hyaluronan production inhibitor fragment is of formula A or B.

In more particular embodiments, the compound of formula Ia is such that the hyaluronan production inhibitor fragment is of formula A.

In such an embodiment, the Y group is selected from

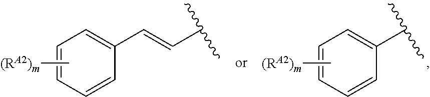

preferably Y is

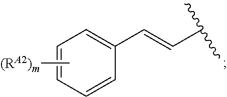

wherein R^{A1}, R^{A2} and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2} or R^{A4} group is

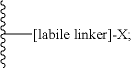

R^{A5} is C₁₋₆ alkyl, C₂₋₆ alkenyl, C₂₋₆ alkynyl, which three groups are optionally substituted by one or more groups selected from halo, —OH and —OMe. Preferably, R^{A5} is C₁₋₆ alkyl, more preferably, R^{A5} is methyl.

In particular embodiments, R^{A1}, R^{A2} and R^{A4} are each independently selected from the group consisting of OH, —OR^{A5}, C₁₋₆ alkyl, C₂₋₆ alkenyl, and C₂₋₆ alkynyl.

In further particular embodiments, R^{A1}, R^{A2} and R^{A4} are each independently selected from the group consisting of OH, —OR^{A5}, C₁₋₆ alkyl, and C₂₋₆ alkenyl.

In further particular embodiments, R^{A1}, R^{A2} and R^{A4} are each independently selected from the group consisting of OH, —OR^{A5} and C₂₋₆ alkenyl.

In further particular embodiments, R^{A1}, R^{A2} and R^{A4} are each independently selected from the group consisting of OH, OMe, and C₅-alkenyl (e.g. prenyl).

In alternative particular embodiments, R^{A1}, R^{A2} and R^{A4} are each independently selected from the group consisting of OH, —OR^{A5}, halo, and C₂₋₆ alkenyl.

In some embodiments, at least two R^{A2} and R^{A4} are at the 3- and 4-positions of the aromatic ring.

In particular embodiments, n is 0 to 5, such as 1 to 4, or 2 to 4.

In particular embodiments, m is 0 to 5, such as 1 to 4, or 2 to 4.

In some embodiments, the labile linker is at the 3- or 4-position of the aromatic ring. Preferably, the labile linker is at the 4-position of the aromatic ring.

In particular embodiments, the compound of formula Ia is such that the hyaluronan production inhibitor fragment is of formula B.

In further particular embodiments, R^{B1}, R^{B2}, and R^{B3} are each independently selected from the group consisting of —OH, —OR^{B4}, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe.

In further particular embodiments, R^{B1}, R^{B2}, and R^{B3} are each independently selected from the group consisting of —OH, —OR^{B4}, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe.

In further particular embodiments, R^{B1}, R^{B2}, and R^{B3} are each independently selected from the group consisting of —OH, —OR^{B4}, C₂₋₆ alkyl, and C₂₋₆alkenyl, which C₁₋₆ alkyl, C₂₋₆ alkenyl groups are optionally substituted with one or more groups selected from halo, —OH and —OMe.

In particular embodiments, R^{B1}, R^{B2}, and R^{B3} are each independently selected from the group consisting of —OH, —OR^{B4}, and C₂₋₆ alkenyl, which C₂₋₆ alkenyl groups are optionally substituted with one or more groups selected from halo, OH and —OMe.

In particular embodiments, R^{B1}, R^{B2}, and R^{B3} are each independently selected from the group consisting of —OH, OMe, and C₅-alkenyl (e.g. prenyl).

In particular embodiments, R^{B1}, R^{B2}, and R^{B3} are each OH.

In particular embodiments, n is 0 to 5, such as 1 to 4, or 2 to 4.

In particular embodiments, m is 0 to 5, such as 1 to 4, or 2 to 4.

In some embodiments, the labile linker is at the 3- or 4-position of the aromatic ring. Preferably, the labile linker is at the 4-position of the aromatic ring.

In particular embodiments, the compound of formula Ia is such that the hyaluronan production inhibitor fragment is of formula C, D or E. In more particular embodiments, the hyaluronan production inhibitor fragment is of formula C. Alternatively, the hyaluronan production inhibitor fragment is of formula D. Alternatively, the hyaluronan production inhibitor fragment is of formula E.

The labile linker is —OC(O)R^{L1}—.

In particular embodiments, R^{L5} is selected from C₁₋₆ alkylene, C₂₋₆ alkenylene, C₂₋₆ alkynylene, which three groups are optionally substituted with one or more groups independently selected from halo, —OR^{L2} or ═O.

In further particular embodiments, R^{L1} is selected from C₁₋₆ alkylene and C₂₋₆ alkenylene, which three groups are optionally substituted with one or more groups independently selected from halo, —OR^{L2} or ═O.

In further particular embodiments, R^{L1} is C₁₋₆ alkylene, such as butylene, propylene, ethylene or methylene, which groups are optionally substituted with one or more groups independently selected from halo, —OR^{L2} or ═O.

In further particular embodiments, R^{L1} is methylene.

In particular embodiments, X is selected from the group consisting of quaternary ammonium, quaternary phosphonium, pyridinium and thiazolium salts.

In particular embodiments, X is selected from the group consisting of:

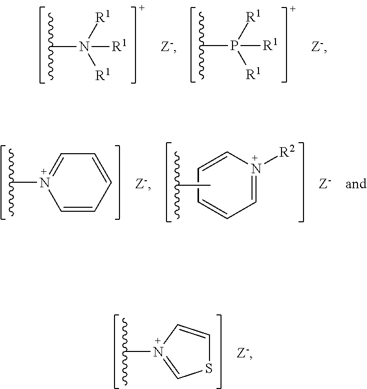

which

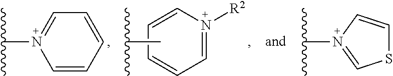

groups are optionally substituted by one or more R³ groups;
In further particular embodiments, X is selected from the group consisting of:

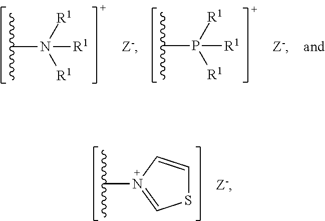

each optionally substituted by one or more R³ groups;
In further particular embodiments, X is

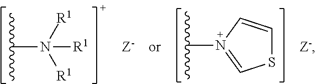

each optionally substituted by one or more R³ groups;

In further particular embodiments, X is

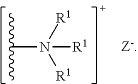

In particular embodiments, R¹ is C₁₋₆ alkyl, optionally substituted with halo or ═O, C₂₋₆ alkenyl, optionally substituted with halo or ═O, C₂₋₆ alkynyl optionally substituted with halo or ═O;

In further particular embodiments, R¹ is C₁₋₆ alkyl, optionally substituted with halo or ═O.

In further particular embodiments, R¹ is selected from the group consisting of n-butyl, n-propyl, ethyl, methyl.

In further particular embodiments, R¹ is methyl.

In particular embodiments, R² is C₁₋₆ alkyl.

In further embodiments, R² is C₁₋₄ alkyl.

In further embodiments, R² is methyl.

In particular embodiments, R³ is C₁₋₆ alkyl, C₂₋₆ alkenyl, C₂₋₆ alkynyl, aryl, heteroaryl.

In further particular embodiments, R³ is C₁₋₆ alkyl, C₂₋₆ alkenyl, C₂₋₆ alkynyl.

In further particular embodiments, R³ is C₁₋₆ alkyl.

In particular embodiments, Z⁻ is selected from the group consisting of Cl⁻, F⁻, Br⁻, I⁻, p-tolylsulphonate, methanesulphonate, acetate, benzoate, salicylate, or R⁴CO₂⁻;

In further particular embodiments, Z is selected from the group consisting Cl⁻, F⁻, Br⁻, I⁻, p-tolylsulphonate, methanesulphonate, acetate, salicylate, or R⁴CO₂⁻.

In further particular embodiments, Z⁻ is Cl⁻, Br⁻ or I⁻.

In alternative embodiments, Z⁻ is selected from the group consisting of Cl⁻, F⁻, Br⁻, I⁻, and R⁴CO₂⁻.

In further embodiments, Z⁻ is R⁴CO₂⁻.

In particular embodiments, R⁴ is C₂₋₂₈ alkyl, C₂₋₂₈ alkenyl, C₂₋₂₈ alkynyl.

In further particular embodiments, R⁴ is C₂₋₂₈ alkenyl.

In further particular embodiments, Z⁻ is the conjugate base of a long chain fatty acid.

As used herein, a long chain fatty acid refers to a straight chain fatty acid (a carboxylic acid with an aliphatic chain) containing twelve or more carbon atoms.

In particular embodiments, Z⁻ is the conjugate base of oleic acid, elaidic acid, vaccenic acid, linoleic acid, linoelaidic acid, ca-linolenic acid, arachidonic acid, erucic acid.

In alternative embodiments, X is

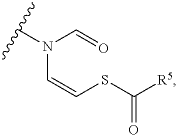

wherein R⁵ is C₁₋₆ alkyl.

In particular embodiments, R⁵ is methyl.

In particular embodiments, the compound of formula Ia is such that:
(i) the hyaluronan production inhibitor fragment is of Formula A, B, C, D or E, such as Formula A or B,
wherein if the hyaluronan production inhibitor fragment is of Formula A, Y is selected from the group consisting of:

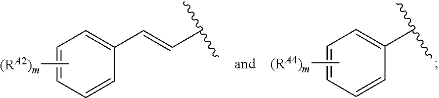

(ii) the labile linker is of formula —OC(O)R^{L1}—, wherein
R^{L1} is selected from the group consisting of C₁₋₆ alkylene, C₂₋₆ alkenylene, C₂₋₆ alkynylene; such as
R^{L1} is selected from the group consisting of C₁₋₆ alkylene and C₂₋₆ alkenylene; or
R^{L1} is selected from the group consisting of butylene, propylene, ethylene, methylene;
(iii) the X group is selected from the group consisting of:

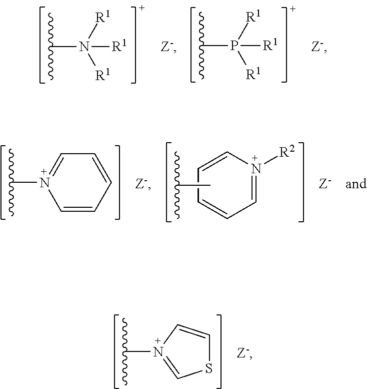

such as

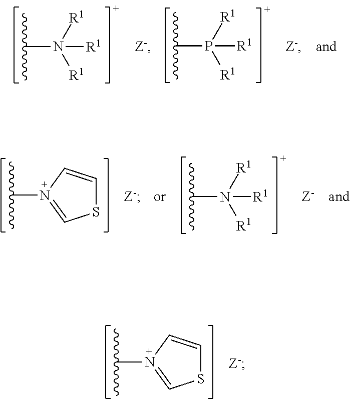

wherein Z⁻ is Cl⁻, F⁻, Br⁻, I⁻, p-tolylsulphonate, methanesulphonate, acetate, benzoate, salicylate, or R⁴CO₂⁻;
such as Z⁻ is Cl⁻, Br⁻ or I⁻ and R⁴CO₂⁻; or
Z⁻ is R⁴CO₂;
wherein R⁴ is C₂₋₂₈ alkyl, C₂₋₂₈ alkenyl, C₂₋₂₈ alkynyl, such as C₂₋₂₈ alkenyl.

In particular embodiments, the compound of formula Ia is such that:
(i) the hyaluronan production inhibitor fragment is of Formula A, B, C, D or E, such as Formula A or B,
wherein if the hyaluronan production inhibitor fragment is of Formula A, Y is selected from the group consisting of;

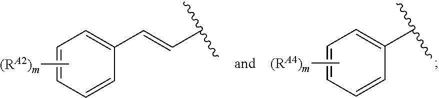

(ii) the labile linker is of formula —OC(O)R^{L1}—, wherein
R^{L1} is selected from the group consisting of C₁₋₆ alkylene, C₂₋₆ alkenylene, C₂₋₆ alkynylene; such as
R^{L1} is selected from the group consisting of C₁₋₆ alkylene and C₂₋₆ alkenylene; or R^{L1} is selected from the group consisting of butylene, propylene, ethylene, methylene;
(iii) X is

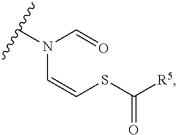

wherein R⁵ is C₁₋₆ alkyl, such as methyl.

Thus, in particular embodiments that may be mentioned, the compound of formula Ia is a compound of formula (II):

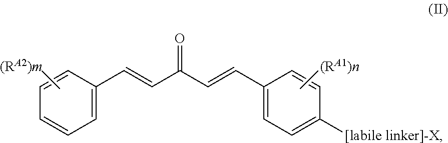

wherein R^{A1}, R^{A2}, X, m, n, and the labile linker are as described herein (i.e. for compounds of the first aspect of the invention, including all embodiments thereof).

In particular such embodiments (i.e. particularly for compounds of formula II), compounds of the invention that may be mentioned include those of formula (IIa):

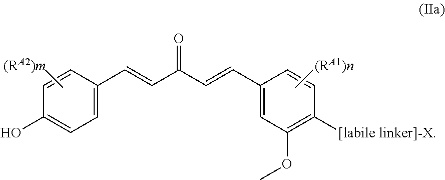

In further particular such embodiments (i.e. particularly for compounds of formula II), compounds of the invention that may also be mentioned include those of formula (IIb):

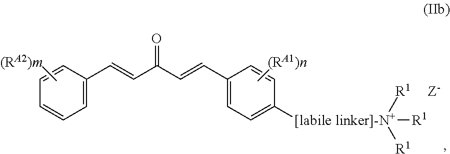

wherein R¹ and Z⁻ are as described herein (i.e. for compounds of the first aspect of the invention, including all embodiments thereof).

In further particular such embodiments (i.e. particularly for compounds of formula II), compounds of the invention that may also be mentioned include those of formula (IIc):

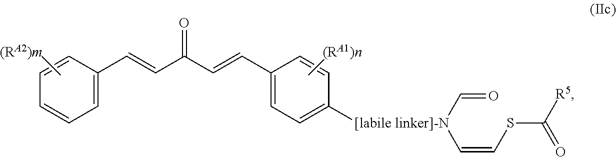

wherein R⁵ is as described herein (i.e. for compounds of the first aspect of the invention, including all embodiments thereof).

In further particular such embodiments (i.e. particularly for compounds of formula II), compounds of the invention that may also be mentioned include those of formula (III):

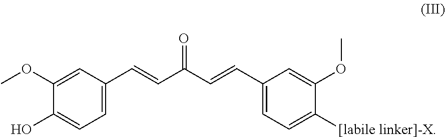

In particular embodiments that may be mentioned, the compound of formula Ia is a compound of formula (VI):

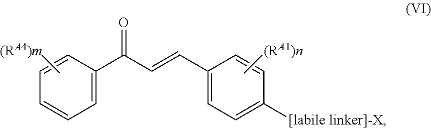

wherein R^{A1}, R^{A4}, X, n, m, and the labile linker are as described herein (i.e. for compounds of the first aspect of the invention, including all embodiments thereof).

In particular such embodiments (i.e. particularly for compounds of formula VI), compounds of the invention that may also be mentioned include those of formula (Via):

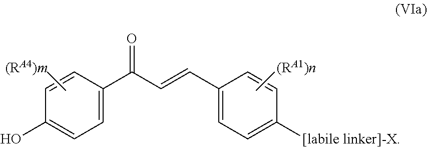

In further particular such embodiments (i.e. particularly for compounds of formula VI), compounds of the invention that may also be mentioned include those of formula (VIb):

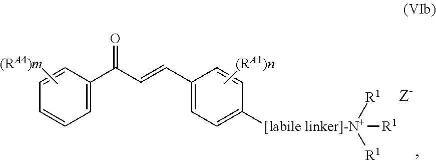

wherein R¹ and Z⁻ are as described herein (i.e. for compounds of the first aspect of the invention, including all embodiments thereof).

In further particular such embodiments (i.e. particularly for compounds of formula VI), compounds of the invention that may also be mentioned include those of formula (VII):

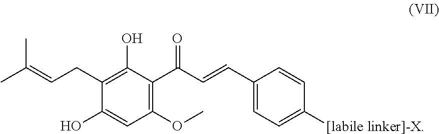

In particular embodiments that may be mentioned, the compound of formula Ia is a compound of formula (VIII):

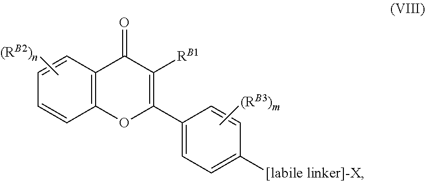

wherein R^{B1}, R^{B2}, R^{B3}, X, n, m, and the labile linker are as described herein (i.e. for compounds of the first aspect of the invention, including all embodiments thereof).

In particular such embodiments (i.e. particularly for compounds of formula VIII), compounds of the invention that may also be mentioned include those of formula (VIIIa):

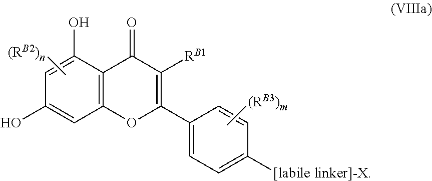

In further particular such embodiments (i.e. particularly for compounds of formula VIII), compounds of the invention that may also be mentioned include those of formula (VIIIb):

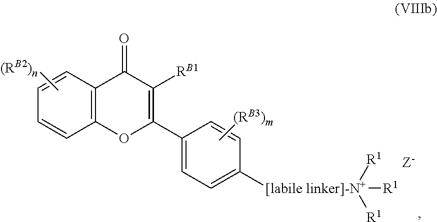

wherein R¹, Z⁻ are as described herein (i.e. for compounds of the first aspect of the invention, including all embodiments thereof).

In further particular such embodiments (i.e. particularly for compounds of formula VIII), compounds of the invention that may also be mentioned include those of formula (VIIIc):

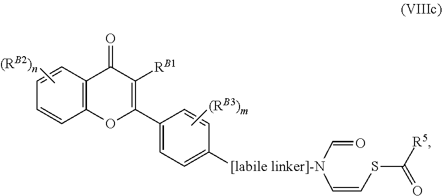

wherein R⁵ is as described herein (i.e. for compounds of the first aspect of the invention, including all embodiments thereof).

In further particular such embodiments (i.e. particularly for compounds of formula VIII), compounds of the invention that may also be mentioned include those of formula (IX):

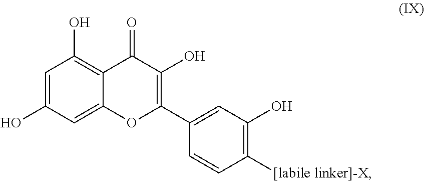

wherein X is as described herein (i.e. for compounds of the first aspect of the invention, including all embodiments thereof).

Particular compounds of the invention that may be mentioned include those compounds as described in the examples provided herein, and pharmaceutically acceptable salts thereof. For the avoidance of doubt, where such compounds of the invention include compounds in a particular salt form, compounds of the invention include those compounds in non-salt form and in the form of any pharmaceutically acceptable salt thereof (which may include the salt form present in such examples).

Thus, particular compounds of the invention that may be mentioned include:

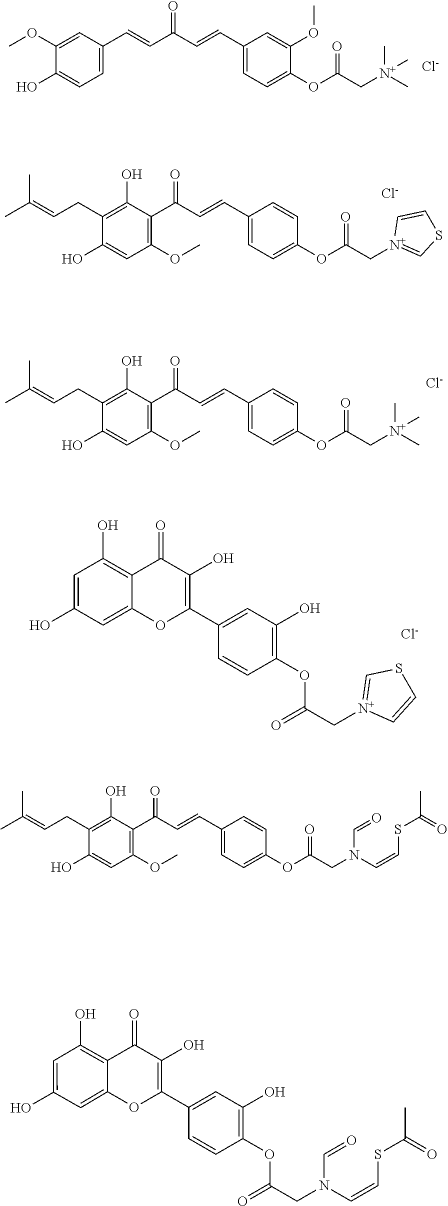

and pharmaceutically acceptable salts thereof.

### Medical Uses

As indicated herein, the compounds of the invention, and therefore compositions and kits comprising the same, are useful as pharmaceuticals.

Thus, according to a second aspect of the invention there is provided a compound of the invention, as hereinbefore defined (i.e. a compound as defined in the first aspect of the invention, including all embodiments and particular features thereof), for use as a pharmaceutical (or for use in medicine).

For the avoidance of doubt, references to compounds as defined in the first aspect of the invention will include references to compounds of formula Ia (including all embodiments thereof, such as compounds of formula II to IX) and pharmaceutically acceptable salts thereof.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

For the avoidance of doubt, compounds of the invention are therefore useful because they possess pharmacological activity, and/or they are metabolised in the body following oral or parenteral administration to form compounds that possess pharmacological activity. In particular, it is believed that the compounds of the invention are useful because they deliver pharmacologically active compounds to their site of action (e.g. cartilage).

As indicated herein, the compounds of the invention may be of use in medicine.

As indicated herein, the compounds of the invention may be of particular use in treating and preventing diseases associated with hyaluronan overproduction, such as rheumatoid diseases.

In a third aspect of the invention, there is provided a compound of formula I:

[hyaluronan production inhibitor]-[labile linker]-X (I)
or a pharmaceutically-acceptable salt thereof,
wherein the hyaluronan production inhibitor fragment is selected from the group consisting of Formula A, B, C, D, and E, wherein formula A is:

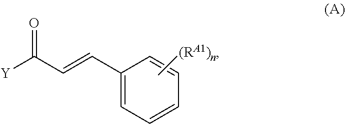

wherein Y is a group selected from the group consisting of:

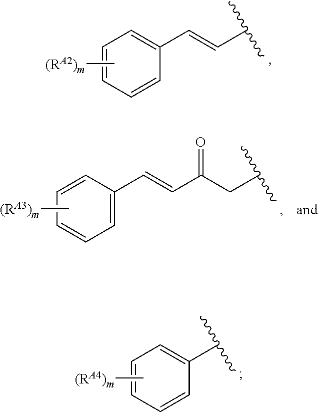

R^{A1}, R^{A2}, R^{A3}, and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2}, R^{A3} or R^{A4} group is

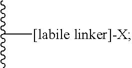

n is 0 to 5;
m is 0 to 5; and
R^{A5} is C₁₋₆ alkyl, C₂₋₆ alkenyl, C₂₋₆ alkynyl, which three groups are optionally substituted by one or more groups selected from halo, —OH and —OMe;
formula B is:

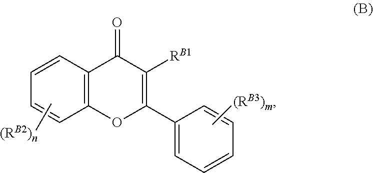

wherein R^{B1}, R^{B2}, and R^{B3} are each independently selected from the group consisting of —OH, —ORB⁴, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe; and one R^{B2} or R^{B3} group is

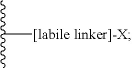

n is 0 to 4;
m is 0 to 5;
R^{B4} is C₁₋₆ alkyl, C₂₋₆ alkenyl, C₂₋₆ alkynyl, which three groups are optionally substituted by one or more groups selected from halo, —OH and —OMe;
formula C is:

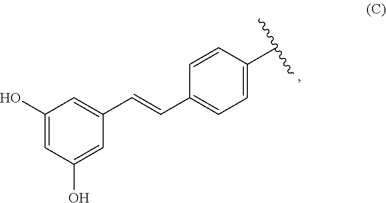 [SMILES: *c1ccc(/C=C/c2cc(O)cc(O)c2)cc1]

wherein the wavy line represents the point of attachment to the labile linker fragment;
formula D is:

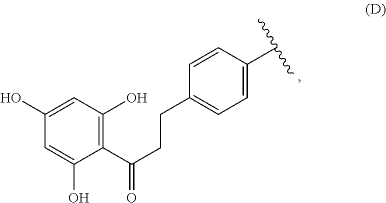 [SMILES: *c1ccc(CCC(=O)c2c(O)cc(O)cc2O)cc1]

wherein the wavy line represents the point of attachment to the labile linker fragment; or
formula E is:

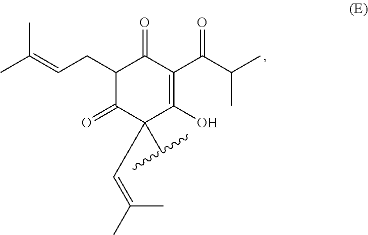 [SMILES: *C1(C=C(C)C)C(=O)C(CC=C(C)C)C(=O)C(C(=O)C(C)C)=C1O]

wherein the wavy line represents the point of attachment to the labile linker fragment;
the labile linker fragment is —OC(O)R^{L1}—;
wherein R^{L1} is selected from C₁₋₆ alkylene, C₂₋₆ alkenylene, C₂₋₆ alkynylene, arylene, or heteroarylene, which five groups are optionally substituted with one or more groups independently selected from halo, —OR^{L2} or ═O;
R^{L2} represents H or C₁₋₆ alkyl;
X is selected from the group consisting of quaternary ammonium, quaternary phosphonium, pyridinium and thiazolium salts; or
X is

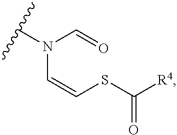

wherein R⁴ is C₁₋₆ alkyl,
for use in the treatment or prevention of a disease associated with hyaluronan overproduction, including rheumatoid diseases.

The compounds for use according to the third aspect of the invention may have any of the features described above for the first aspect of the invention (including all embodiments thereof, such as compounds of formula I to IX).

The compounds of formula I may be referred to as “compounds of the third aspect of the invention”.

In particular embodiments that may be mentioned, the compound for use according to the third aspect of the invention, is a compound of formula (IV):

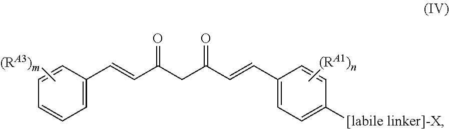

wherein R^{A1}, R^{A3}, X, n, m, and the labile linker are as described herein (i.e. for compounds of the first aspect of the invention, including all embodiments thereof).

In particular embodiments, R^{A1} and R^{A3} are each independently selected from the group consisting of OH, —OR^{A5}, C₁₋₆ alkyl, C₂₋₆ alkenyl, and C₂₋₃ alkynyl.

In further particular embodiments, R^{A1} and R^{A3} are each independently selected from the group consisting of OH, —OR^{A5}, C₁₋₆ alkyl, and C₂₋₆ alkenyl.

In further particular embodiments, R^{A1} and R^{A3} are each independently selected from the group consisting of OH, —OR^{A5}, and C₂₋₆ alkenyl.

In further particular embodiments, R^{A1} and R^{A3} are each independently selected from the group consisting of OH, OMe, and C₅-alkenyl (e.g. prenyl).

In alternative particular embodiments, R^{A1} and R^{A3} are each independently selected from the group consisting of OH, —OR^{A5}, halo, and C₂₋₆ alkenyl.

In some embodiments, m is at least two (e.g. two) and the R^{A2} groups are at the 3- and 4-positions of the aromatic ring.

In particular such embodiments (i.e. particularly for compounds of formula IV), compounds of the invention that may also be mentioned include those of formula (IVa):

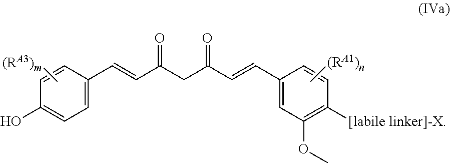

In further particular such embodiments (i.e. particularly for compounds of formula IV), compounds of the invention that may also be mentioned include those of formula (V):

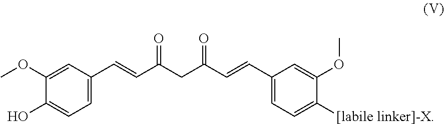

Particular diseases and disorders associated with hyaluronan overproduction that may be mentioned are rheumatoid diseases, inflammation, cancer, metastasis, heart infarction, kidney dysfunction, stroke, fibrosis, ischemia reperfusion injury, autoimmune diseases, chemoresistance, type 1 diabetes (T1D), multiple sclerosis, liver cirrhosis, asthma, Werner's syndrome, cutaneous hyaluronanosis.

Preferably, the disease is a rheumatoid disease, inflammation, cancer, metastasis, heart infarction, kidney dysfunction, stroke, fibrosis, ischemia reperfusion injury or an autoimmune disease.

Particular rheumatoid diseases that may be mentioned are ankylosing spondylitis, bursitis, Crohn's disease, gout, haemophilia, infectious arthritis, inflammatory muscle disease, juvenile idiopathic arthritis, neuropathic arthropathy, osteoarthritis, osteoporosis, osteonecrosis, polymyalgia rheumatica (PMR), polymyositis, psoriatic arthritis, rheumatoid arthritis (RA), scleroderma, skeletal hyperostosis, Sjogren's syndrome, spondyloarthritis, spondyloarthropathies, systemic lupus erythematosus, systemic sclerosis, tendinitis vasculitis, von Willebrand disease.

In preferred embodiments of the invention, the compounds of the third aspect of the invention (i.e. compounds of formula I) are for use in treating or preventing rheumatoid diseases. The rheumatoid disease may be rheumatoid arthritis, osteoarthritis, infectious arthritis, spondyloarthritis, psoriatic arthritis, Sjogren's syndrome, systemic lupus erythematosus, systemic sclerosis, inflammatory muscle disease, vasculitis, gout, skeletal hyperostosis, neuropathic arthropathy, osteonecrosis, haemophilia, von Willebrand disease.

In more preferred embodiments of the invention, the disease is rheumatoid arthritis, osteoarthritis or infectious arthritis.

In yet more preferred embodiments of the invention, the disease is osteoarthritis.

In an alternative third aspect of the invention, there is provided a method of treating and preventing a disease associated with hyaluronan overproduction, such as rheumatoid diseases, inflammation, cancer, metastasis, heart infarction, kidney dysfunction, stroke, fibrosis, ischemia reperfusion injury, autoimmune diseases, chemoresistance, type 1 diabetes (T1D), multiple sclerosis, liver cirrhosis, asthma, Werner's syndrome, cutaneous hyaluronanosis, comprising administering to a patient in need thereof a therapeutically effective amount of a compound of the third aspect of the invention (i.e. a compound of formula I), as hereinbefore defined.

In a further alternative third aspect of the invention, there is provided the use of a compound of the third aspect of the invention (i.e. a compound of formula I), as hereinbefore defined, for the manufacture of a medicament for the treatment and prevention of a disease or disorder associated with hyaluronan overproduction, such as rheumatoid diseases, inflammation, cancer, metastasis, heart infarction, kidney dysfunction, stroke, fibrosis, ischemia reperfusion injury, autoimmune diseases, chemoresistance, type 1 diabetes (T1D), multiple sclerosis, liver cirrhosis, asthma, Werner's syndrome, cutaneous hyaluronanosis.

For the avoidance of doubt, the term “hyaluronan overproduction” as used herein will be understood by those skilled in the art to refer to an excessive amount of hyaluronan in the synovial fluid or serum of the subject experiencing the same. In particular, it may refer to a subject (eg. a human subject) having hyaluronan serum levels higher than 56.0±16.0 μg/L The serum levels in healthy humans are 56.0±16.0 μg/L. The hyaluronan concentration is elevated in osteoarthritis and rheumatoid arthritis and remission is associated with the reversal of these changes (Sasaki E et al, Int Orthop. 2013. 37(5):925-30; Pothacharoen P et al, Osteoarthritis Cartilage. 2006. 14(3):299-301; Pitsillides A A et al, Arthritis Rheum., 1994 July:37(7):1030-8). The serum levels in patients with osteoarthritis are 104.9±16 μg/L, while in patients with rheumatoid arthritis the serum levels are 197.1±122.9 μg/L. In certain patients, the hyaluronan excessive overproduction may lead to an increase in the volume of the synovial cavity, and in some cases the excess hyaluronan may need to be physically extracted.

The concentration of hyaluronan can be determined by appropriate techniques as known to those skilled in the art, such as ELISA.

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms. For example, in the case of osteoarthritis, the term may refer to a reduction in pain and slowing of further progression of joint cartilage damage.

As used herein, the term prevention (and, similarly, preventing) will include references to the prophylaxis of the disease or disorder (and vice-versa). As such, references to prevention may also be references to prophylaxis, and vice versa. In particular, such terms term may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

As used herein, references to a patient (or to patients) will refer to a living subject being treated, including mammalian (e.g. human) patients. In particular, references to a patient will refer to human patients.

For the avoidance of doubt, the skilled person will understand that such treatment or prevention will be performed in a patient (or subject) in need thereof. The need of a patient (or subject) for such treatment or prevention may be assessed by those skilled the art using routine techniques.

As used herein, the terms disease and disorder (and, similarly, the terms condition, illness, medical problem, and the like) may be used interchangeably.

As used herein, the term effective amount will refer to an amount of a compound that confers a therapeutic effect on the treated patient. The effect may be observed in a manner that is objective (i.e. measurable by some test or marker) or subjective (i.e. the subject gives an indication of and/or feels an effect). In particular, the effect may be observed (e.g. measured) in a manner that is objective, using appropriate tests as known to those skilled in the art. For example, for osteoarthritis, in humans, the distance of the synovial cleft or joint space narrowing and osteophytogenesis or subchondral sclerosis may be analysed by X-ray. More detailed erosion may be visualised by MRI.

In particular embodiments, compounds of the third aspect of the invention (i.e. compounds of formula I, including all embodiments thereof) are for use in the treatment of diseases associated with hyaluronan overproduction (or useful in the manufacture of a medicament for such treatment, or useful in a method for such treatment, as described herein).

In particular embodiments of the third aspect of the invention, the disease is a rheumatoid disease, such as a rheumatoid disease selected from the list consisting of rheumatoid arthritis, osteoarthritis, infectious arthritis, spondyloarthritis, psoriatic arthritis, Sjogren's syndrome, systemic lupus erythematosus, systemic sclerosis, inflammatory muscle disease, vasculitis, gout, skeletal hyperostosis, neuropathic arthropathy, osteonecrosis, haemophilia, von Willebrand disease.

In particular embodiments, the treatment may be of a disease associated with hyaluronan overproduction in a patient who is at risk of developing a rheumatoid disease. Thus, compounds of the third aspect of the invention (i.e. compounds of formula I) may be useful in the prevention of a rheumatoid disease.

As indicated herein, the compounds of the third aspect of the invention may find particular utility in the prevention of osteoarthritis after traumatic injuries which often occur during physical activity (such as tennis, soccer, extensive jogging), or in the case of overweight or elderly persons.

In particular embodiments, the compounds of the third aspect of the invention (i.e. compounds of formula I) are for use in the treatment or prevention of cancer.

In some embodiments, the cancer is a soft tissue cancer, such as sarcoma (e.g. chondrosarcoma, angiosarcoma, fibrosarcoma, rhabdomyosarcoma, liposarcoma), myxoma, rhabdomyoma, fibroma, lipora and teratoma.

In preferred embodiments, the cancer is sarcoma. More preferably, the cancer is chondrosarcoma.

In particular embodiments, the treatment of a disease associated with hyaluronan overproduction as defined herein and including, particularly, rheumatoid arthritis and osteoarthritis, comprises administering the compound of formula I or Ia (including pharmaceutically acceptable salts thereof) or pharmaceutical composition comprising the same, intravenously, topically, intraarticularly (i.e. via intra-articular administration), transdermally (e.g. by iontophoresis), or orally.

More particularly, the treatment of a disease associated with hyaluronan overproduction as defined herein and including, particularly, rheumatoid arthritis and osteoarthritis, comprises administering the compound of formula I or Ia (including pharmaceutically acceptable salts thereof) or pharmaceutical composition comprising the same) intravenously, topically, intraarticularly (i.e. via intraarticular administration) or transdermally (e.g. by iontophoresis).

In particular embodiments, the compound of the invention is administered intravenously.

In particular embodiments, the compound of the invention is administered topically.

In particular embodiments, the compound of the invention is administered intraarticularly.

In particular embodiments, the compound of the invention is administered transdermally.

In particular embodiments that may be mentioned, there is provided a compound of the invention (i.e. a compound of formula I, or Ia, as defined herein, including pharmaceutically acceptable salts thereof) for use in the treatment of a disease associated with hyaluronan overproduction as defined herein, and including, particularly, rheumatoid arthritis and osteoarthritis wherein the compound is administered intraarticularly, for example to the individual affected joint(s).

### Pharmaceutical Compositions

As described herein, compounds of the invention are useful as pharmaceuticals. Such compounds may be administered alone or may be administered by way of known pharmaceutical compositions/formulations.

In a fourth aspect of the invention, there is provided a pharmaceutical composition comprising a compound of the first aspect of invention as defined herein (i.e compounds of formula Ia), and optionally one or more pharmaceutically-acceptable excipient.

As used herein, the term pharmaceutically-acceptable excipients includes references to vehicles, adjuvants, carriers, diluents, pH adjusting and buffering agents, tonicity adjusting agents, stabilizers, wetting agents and the like. In particular, such excipients may include adjuvants, diluents or carriers.

In a particular embodiment of the fourth aspect of the invention, the pharmaceutical composition comprises at least one pharmaceutically-acceptable excipient.

For the avoidance of doubt, references herein to compounds of invention being for particular uses (and, similarly, to uses and methods of use relating to compounds of the invention) may also apply to pharmaceutical compositions comprising compounds of the invention, as described herein.

Thus, in a fifth aspect of the invention, there is provided a pharmaceutical composition as defined in the fourth aspect of the invention for use in the treatment or prevention of a disease characterised by hyaluronan overproduction (as defined herein, with reference to the third aspect of the invention and all embodiments thereof).

The skilled person will understand that compounds of the invention may act systemically and/or locally (i.e. at a particular site), and may therefore be administered accordingly using suitable techniques known to those skilled in the art. The skilled person will understand that compounds and compositions as described herein will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form.

Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration (including for iontophoresis).

Thus, in particular embodiments, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In particular embodiments, the compounds of the invention are administered intravenously, and pharmaceutical compositions of the invention may therefore be formulated for intravenous administration. This route of administration is preferable as it is fast and ensures complete bioavailability. The compounds of the invention are particularly suitable for intravenous administration.

Alternatively, the compounds of the invention are administered topically and pharmaceutical compositions of the invention may be formulated for topical administration. More particularly, the compounds of the invention are administered by intra-articular administration and the compositions of the invention may be formulated for intra-articular administration.

Intra-articular administration (such as direct injection into the synovial fluid) may be particularly suitable for patients with individual joints being affected by the disease, such as arthritis (e.g. osteoarthritis) patients with one or more knees and/or hips being affected.

In further particular embodiments, the compounds of the invention are administered transdermally and the compositions formulated for transdermal administration, including particularly by iontophoresis.

For topical administration, pharmaceutical compositions of the present invention may be encapsulated in monooleine aqueous dispersion (Puglia C. et al, J. Pharm Sci, 2013, 102(7), 2349-2361).

Alternatively, for topical administration, the compounds of the invention may be used in a hydrophobic ion pair delivery system (Al-Dujaili E. A. Indian Drugs 2020. 57 (01), 7-18; Torky A. S. et al, Int J Pharm, 2018, 76-86), preferably wherein the ion pair complexing agent is oleic acid (Lu et al, Mol. Pharm, 2018, 216-225).

The pharmaceutical compositions of the invention may also be formulated for oral administration.

Depending on e.g. potency and physical characteristics of the compound of the invention (i.e. active ingredient), pharmaceutical formulations that may be mentioned include those in which the active ingredient is present in an amount that is at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The skilled person will understand that compounds of the invention may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 25 mg, 50 mg, 100 mg or 200 mg twice daily).

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

For the avoidance of doubt, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. Although the above-mentioned dosages are exemplary of the average case, there can, of course, be individual instances where higher or lower dosage ranges are merited, and such doses are within the scope of the invention.

### Combinations and Kits-of-Parts

The skilled person will understand that treatment with compounds of the invention may further comprise (i.e. be combined with) further treatment(s) or preventative methods for the same condition. In particular, treatment with compounds of the invention may be combined with means for the treatment of a disease associated with hyaluronan overproduction (such as a type of rheumatoid disease as described herein, e.g. arthritis or osteoarthritis), such as treatment with one or more other therapeutic agent that is useful in the in the treatment or prevention of the disease associated with hyaluronan overproduction and/or one or more physical method used in the treatment or prevention of the disease associated with hyaluronan overproduction (such as treatment through surgery), as known to those skilled in the art.

As described herein, compounds of the invention may also be combined with one or more other (i.e. different) therapeutic agents (i.e. agents that are not compounds of the invention) that are useful in the treatment or prevention of a disease associated with hyaluronan overproduction. Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a sixth aspect of the invention, there is provided a combination product comprising:
(I) a compound of the invention, as hereinbefore defined (i.e. in the first aspect of the invention, including all embodiments and particular features thereof); and
(II) one or more other therapeutic agent that is useful in the treatment or prevention a disease associated with hyaluronan overproduction (such as a rheumatoid disease as described herein),
wherein each of components (I) and (II) is formulated in admixture, optionally with one or more a pharmaceutically-acceptable excipient.

In a seventh aspect of the invention, there is provided a kit-of-parts comprising:
(a) a pharmaceutical formulation as hereinbefore defined (i.e. in the fifth aspect of the invention); and
(b) one or more other therapeutic agent that is useful in the treatment or prevention of a disease associated with hyaluronan overproduction (such as a rheumatoid disease as described herein), optionally in admixture with one or more pharmaceutically-acceptable excipient,
which components (a) and (b) are each provided in a form that is suitable for administration in conjunction (i.e. concomitantly or sequentially) with the other.

With respect to the kits-of-parts as described herein, by “administration in conjunction with” (and similarly “administered in conjunction with”) we include that respective formulations are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients (i.e. a compound of the invention and a further agent for the treatment of the disease associated with hyaluronan overproduction, or compositions comprising the same) are administered (optionally repeatedly) either together, or sufficiently closely in time, to enable a beneficial effect for the patient, that is greater, over the course of the treatment or prevention of the relevant condition, than if either agent is administered (optionally repeatedly) alone, in the absence of the other component, over the same course of treatment or prevention. Determination of whether a combination provides a greater beneficial effect in respect of, and over the course of, treatment or prevention of a particular condition will depend upon the condition to be treated or prevention, but may be achieved routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of the disease associated with hyaluronan overproduction, or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

As used herein, references to other therapeutic agents that are “useful” in a certain manner (e.g. in the treatment of a certain disease or disorder) will refer to agents that are known to be suitable for use in that manner (e.g. agents commonly used for that purpose). Such references may therefore be replaced with references to agents “suitable for” the relevant purpose.

Other therapeutic agents useful in the treatment or prevention of diseases associated with hyaluronan overproduction (such as rheumatoid diseases described herein) will be well-known to those skilled in the art. For example, such other therapeutic agents may include inhibitors of inflammation such as aspirin, ibuprofen, naproxen, meloxicam, celecoxib, indomethacin, corticosteroids (such as prednisone), antimalarial medication (such as hydroxychloroquine). Other therapeutic agents may include disease-modifying antirheumatic drugs (DMARDs) such as methotrexate, sulfasalazine, leflunornide, azathioprine, and cyclophosphamide. Other therapeutic agents may also include biologic drugs such as infliximab, etanercept, adalimurnab, certolizurnab, golimumab, abatacept, tocilizumab, and rituximab.

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable excipient.

In further aspects of the invention, there is provided a process for the preparation of a combination product or kit-of-parts as hereinbefore defined, which process comprises bringing into association a compound of the invention, as hereinbefore defined, with the other therapeutic agent that is useful in the treatment of the relevant disease or disorder, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention as described herein may be prepared in accordance with techniques that are well known to those skilled in the art, such as those described in the examples provided hereinafter.

According to an eighth aspect of the invention there is provided a process for the preparation of a compound of the invention as hereinbefore defined (i.e. a compound of Formula Ia), which process comprises the step of reacting a compound of formula (XI),

[hyaluronan production inhibitor]-[labile linker]-LG¹ (XI),
wherein the hyaluronan production inhibitor fragment and the labile linker are as hereinbefore defined and LG¹ is selected from the group consisting of chloro, bromo, fluoro, iodo, and a protected alcohol (such as tosylate, mesylate, acetate) with an organic nucleophile (Nu) selected from the group consisting of a tertiary amine, a tertiary phosphine, a pyridine and a thiazole.

The reaction may be performed, for example, in a suitable solvent (such as THF), at a suitable reaction temperature (e.g. between room temperature and reflux temperature).

In particular embodiments, Nu is selected from the group consisting of

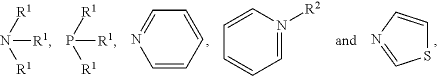

which

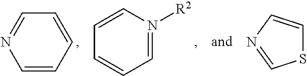

groups are optionally substituted by one or more R³ groups;
R¹ is C₁₋₆ alkyl, optionally substituted with halo or ═O, C₂₋₆ alkenyl, optionally substituted with halo or ═O, C₂₋₆ alkynyl, optionally substituted with halo or ═O;
R² is C₁₋₆ alkyl;
R³ is C₁₋₆ alkyl, C₂₋₆ alkenyl, C₂₋₆ alkynyl, aryl, heteroaryl.

Compounds of formula XI may be obtained by reacting a hyaluronan production inhibitor, wherein the hyaluronan production inhibitor is as defined hereinbefore, with a compound of formula XII containing an acyl chloride (such as chloroacetyl chloride),

CIC(O)R^{L1}-LG¹ (XII),
wherein R^{L1} is as defined hereinbefore.

LG¹ is selected from the group consisting of chloro, bromo, fluoro, iodo, and a protected alcohol (such as tosylate, mesylate, acetate).

The reaction may be performed, for example, in a suitable solvent (such as THF), in the presence of a suitable base (such as pyridine) at a suitable reaction temperature (e.g. between room temperature and reflux temperature).

In this step, the reaction of a compound of the hyaluronan production inhibitor with the compound of formula XII forms the ester of the labile linker fragment through reaction of a hydroxyl group on the hyaluronan production inhibitor with the acyl chloride of the compound of formula XII.

Alternatively, in this aspect of the invention, there is provided a process for the preparation of a compound of the invention as hereinbefore defined (i.e. a compound of Formula Ia), comprising the steps of reacting a hyaluronan production inhibitor compound as hereinbefore defined, with a compound of formula XIII,

CIC(O)R^{L1}—X (XIII),
wherein R^{L1} and X are defined herein before.

The reaction may be performed, for example, in a suitable solvent (such as pyridine), at a suitable reaction temperature (e.g. between room temperature and reflux temperature).

In this step, the reaction of a compound of the hyaluronan production inhibitor with the compound of formula XIII forms the ester of the labile linker fragment through reaction of a hydroxyl group on the hyaluronan production inhibitor with the acyl chloride of the compound of formula XIII.

Compounds of formula XIII may be obtained by reacting a compound of formula XIV,

HO—C(O)R^{L1}—X (XIV)
wherein R^{L1} and X are as defined hereinbefore, with a chlorinating agent (such as thionyl chloride).

The reaction may be performed, for example, in a suitable solvent or in the absence of solvent (e.g. in neat reagents), at a suitable reaction temperature (e.g. between room temperature and reflux temperature).

The starting materials in the Examples below are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

The skilled person will understand that the substituents as defined herein, and substituents thereon, may be modified one or more times, after or during the processes described above for the preparation of compounds of the invention by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula Ia, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis” 3rd edition, T. W. Greene & P. G. M. Wutz, Wiley-Interscience (1999), the contents of which are incorporated herein by reference.

Compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### BRIEF DESCRIPTION OF THE FIGURES

FIG. 1 shows the results of an Alamar blue toxicity assay, showing that xanthohumol (X), xanthohumol-betaine (X-B), xanthohumol-prothiazole (X-Pro), quercetin (Q) and quercetin-prothiazole (Q-Pro) were not toxic below concentrations of 100 μM.

FIGS. 2A and 2B show the uptake of flavonoids into chondrosarcoma cells, by intracellular fluorescence, in a concentration (A) and time (B) dependent manner. FIG. 2A shows that the derivatised flavonoids (X-Pro, Q-Pro and X-B) were taken up in a concentration dependent manner, whereas the original xanthohumol and quercetin did not accumulate inside the cells. FIG. 2B shows that the uptake of the derivatised flavanoids was initially very rapid and reached a plateau after 10 hours.

FIG. 3 shows the effect of the compounds on chondrosarcoma cell growth. The compounds inhibited cell growth in a dose-dependent manner.

FIG. 4 shows the effect of the compounds in inhibiting hyaluronan export.

FIGS. 5A and 5B show the inhibition of proteoglycan shedding from bovine cartilage slices by xanthohumol (FIG. 5A) and xanthohumol-betaine (FIG. 5B).

FIG. 6A to 6E show the inhibition of proteoglycan loss by xanthohumol-betaine (FIG. 6A), xanthohumol-prothiazole (FIG. 6B), quercetin-prothiazole (FIG. 6C), quercetin (FIG. 6D), xanthohumol (FIG. 6E).

FIGS. 7A to 7E show the concentration dependency of xanthohumol (FIG. 7A), xanthohumol-prothiazole (FIG. 7B), xanthohumol-betaine (FIG. 7C), quercetin (FIG. 7D), quercetin-prothiazole (FIG. 7E) on collagen loss from bovine cartilage slices.

FIGS. 8A and 8B show that hylin-betaine (Compound of Example 1) and xantho-betaine (Compound of Example 2) treatments do not alter metaphyseal trabecular bone. Trabecular bone phenotype of vehicle, hylin-betaine and xantho-betaine treated Str/ort mice. (A) Representative 3D Micro-CT thickness colour-coded images of tibial trabecular 25 bone. Ex vivo high-resolution analyses of distal proximal metaphysical tibia to determine (B) bone length, trabecular bone volume/total volume, trabecular TV, trabecular BV, trabecular eccentricity, trabecular thickness, separation and number. Graphs represent means t SEM. Group sizes for Micro-CT (vehicle and hylin-betaine) were n=6 and n=5 for xantho-betaine treated mice. One-way ANOVA with Bonferroni post-hoc analysis between groups were performed. Normality of variance assumption and homogeneity were not violated for any of the parameters.

FIG. 9 shows that hylin-betaine (Compound of Example 1) and xantho-betaine (Compound of Example 2) treatments significantly alter cortical bone. (A) Bone mean cortical thickness and (B) cross-sectional area (CSA) of vehicle, hylin-betaine and xanto treated Str/ort mice between 10-90% of total tibial length excluding proximal and distal metaphyseal bone which contain trabecular bone. Line graphs represent means±SEM. Group sizes were n=6 for vehicle and hylin-betaine and 5 for xantho-betaine group. Graphical heat map summarises statistical differences at specific matched locations along the tibial length, representation of the overall effect of treatment and Bonferroni post-hoc analysis are also shown

FIG. 10 shows that hylin-betaine (Compound of Example 1) and xantho-betaine (Compound of Example 2) treatments significantly alter cortical bone. (A) Minimum and (B) maximum second moments of area (Iₘᵢₙ and Iₘₐₓ respectively), (C) J (resistance to torsion) and (D) ellipticity of vehicle, hylin-betaine and xantho-betaine treated Str/ort mice. Whole bone analyses of cortical bone between 10-90% of total tibial length, excluding proximal and distal metaphyseal bone. Line graphs represent means±SEM. Group sizes were n=6 for vehicle and hylin-betaine and 5 for xantho-betaine group. Graphical heat map summarises statistical differences at specific matched locations along the tibial length, representative of overall effect of treatment and Bonferroni post-hoc analysis are also shown.

FIG. 11 shows that hylin-betaine (H) but not xantho-betaine (X) treatment prevents/improves articular cartilage lesions in Str/ort mice. Sections were stained with toluidine blue and scored according to OARSI scoring system. Vehicle and treated groups exhibited localized lesions. Mean and maximum±SEM lesion severity scores in each compartment of vehicle joints and treated joints. * denotes statistical significance p<0.05

For the avoidance of doubt, the compounds hylin, hylin-betaine, xanthohumol-betaine (X-B), xanthohumol-prothiazole (X-Pro), quercetin-prothiazole (Q-Pro) used in figure legends refer to the compounds in the examples as provided herein.

### Aspects

Certain aspects and embodiments of the present invention are set out in the following numbered paragraphs:

1. A compound of Formula Ia:

[hyaluronan production inhibitor]-[labile linker]-X (Ia),
or a pharmaceutically acceptable salt thereof,
wherein the hyaluronan production inhibitor fragment is of:
formula A

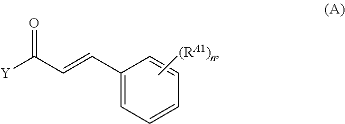

wherein Y is a group selected from the group consisting of:

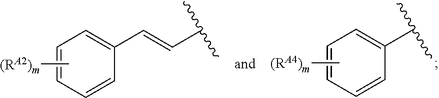

R^{A1}, R^{A2}, and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2} or R^{A4} group is

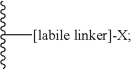

n is 0 to 5;
m is 0 to 5; and
R^{A5} is C₁₋₆ alkyl, C₂₋₆ alkenyl, C₂₋₆ alkynyl, which three groups are optionally substituted by one or more groups selected from halo, —OH and —OMe;
formula B

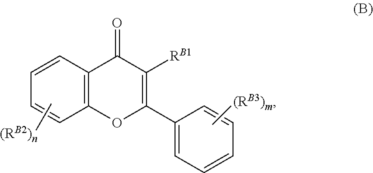

wherein R^{B1}, R^{B2}, and R^{B3} are each independently selected from the group consisting of —OH, —OR^{B4}, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe; and one R^{B2} or R^{B3} group is

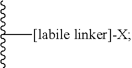

n is 0 to 4;
m is 0 to 5;
R^{B4} is C₁₋₆ alkyl, C₂₋₆ alkenyl, C₂₋₆ alkynyl, which three groups are optionally substituted by one or more groups selected from halo, —OH and —OMe;
formula C

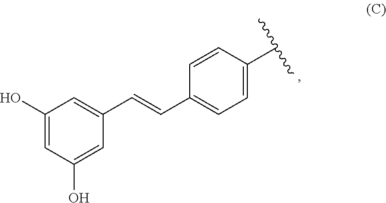 [SMILES: *c1ccc(/C=C/c2cc(O)cc(O)c2)cc1]

wherein the wavy line represents the point of attachment to the labile linker fragment;
formula D

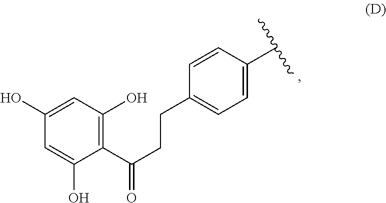 [SMILES: *c1ccc(CCC(=O)c2c(O)cc(O)cc2O)cc1]

wherein the wavy line represents the point of attachment to the labile linker fragment; or
formula E

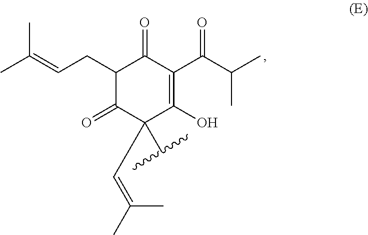 [SMILES: *C1(C=C(C)C)C(=O)C(CC=C(C)C)C(=O)C(C(=O)C(C)C)=C1O]

wherein the wavy line represents the point of attachment to the labile linker fragment;
the labile linker fragment is —OC(O)R^{L1}—;
wherein R^{L1} is selected from C₁₋₆ alkylene, C₂₋₆ alkenylene, C₂₋₆ alkynylene, arylene, or heteroarylene, which five groups are optionally substituted with one or more groups independently selected from halo, —OR^{L2} or ═O;
R^{L2} represents H or C₁₋₆ alkyl;
X is selected from the group consisting of quaternary ammonium, quaternary phosphonium, pyridinium and thiazolium salts; or
X is

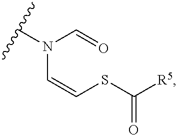

wherein R⁵ is C₁₋₆ alkyl.

2. A compound according to Paragraph 1, wherein the hyaluronan production inhibitor fragment is of formula A or B.

3. A compound according to any one of the preceding paragraphs, wherein the compound is of formula II:

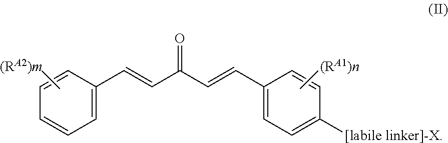

4. A compound according to any one of the preceding paragraphs, wherein the compound is of formula III:

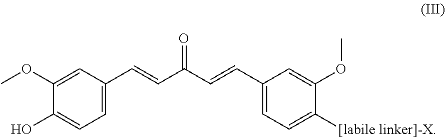

5. A compound according to any one of the preceding paragraphs, wherein the compound is:

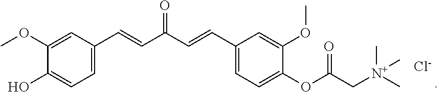

6. A compound according to any one of Paragraphs 1 to 2, wherein the compound is of formula (VI):

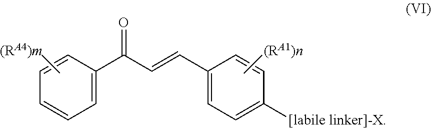

7. A compound according to Paragraph 6, wherein the compound is of formula (VII):

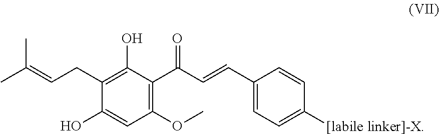

8. A compound according to Paragraph 7, wherein the compound is

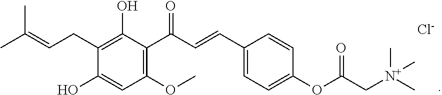 [SMILES: COc1cc(O)c(CC=C(C)C)c(O)c1C(=O)/C=C/c1ccc(OC(=O)C[N+](C)(C)C)cc1 ; [Cl-]]

9. A compound according to Paragraph 7, wherein the compound is:

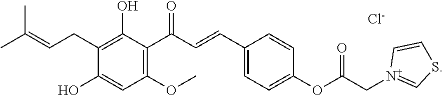 [SMILES: COc1cc(O)c(CC=C(C)C)c(O)c1C(=O)/C=C/c1ccc(OC(=O)C[n+]2ccsc2)cc1 ; [Cl-]]

10. A compound according to Paragraphs 1 or 2, wherein the compound is of formula VIII:

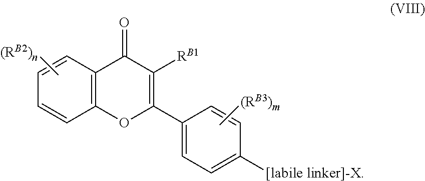

11. A compound according to Paragraph 10, wherein the compound is of Formula IX:

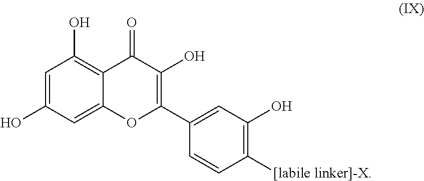

12. A compound according to Paragraph 11, wherein the compound is:

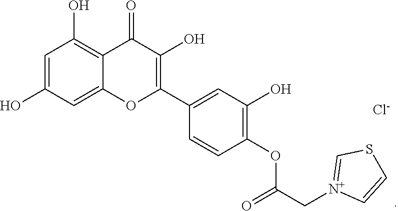 [SMILES: O=C(C[n+]1ccsc1)Oc1ccc(-c2oc3cc(O)cc(O)c3c(=O)c2O)cc1O ; [Cl-]]

13. A compound according to Paragraph 1 wherein the hyaluronan production inhibitor fragment is of formula C, D or E.

14. A compound according to any one of Paragraphs 1 to 4, 6, 7, 10, 11, or 13, wherein X is selected from the group consisting of:

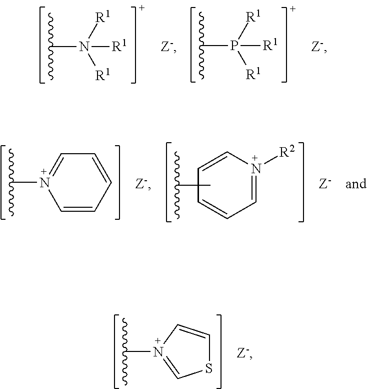

which

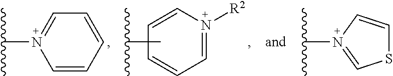

groups are optionally substituted by one or more R³ groups;
R¹ is C₁₋₆ alkyl, optionally substituted with halo or ═O, C₂₋₆ alkenyl, optionally substituted with halo or ═O, C₂₋₆ alkynyl, optionally substituted with halo or ═O;
R² is C₁₋₆ alkyl;
R³ is C₁₋₆ alkyl, C₂₋₆ alkenyl, C₂₋₆ alkynyl, aryl, heteroaryl;
Z⁻ is selected from the group consisting of Cl⁻, F⁻, Br⁻, I⁻, p-tolylsulphonate, methanesulphonate, acetate, benzoate, salicylate, or R⁴CO₂;
R⁴ is C₂₋₂₈ alkyl, C₂₋₂₈ alkenyl, C₂₋₂₈ alkynyl.

15. A compound according to Paragraph 14, wherein the X group is

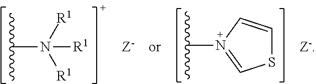

16. A compound according to any one of Paragraphs 14 or 15, wherein the Z⁻ group is R⁴CO₂—, wherein R⁴ is C₄₋₂₈ alkyl or C₄₋₂₃ alkenyl.

17. A compound according to Paragraph 1, wherein R^{A1} to R^{A4} and R^{B1} to R^{B3} are selected from the group consisting of OH, OMe, C₂₋₆ alkenyl.

18. A pharmaceutical composition comprising a compound as defined in any one of Paragraphs 1 to 17, including a pharmaceutically acceptable salt thereof, and optionally one or more pharmaceutically-acceptable excipient.

19. A compound according to any one of Paragraphs 1 to 17 or a pharmaceutical composition according to Paragraph 18 for use as a medicament.

20. A compound according to any one of Paragraphs 1 to 17 or a pharmaceutical composition according to Paragraph 18 for use in the treatment or prevention of disease characterised by hyaluronan overproduction.

21. A compound according to any one of Paragraphs 1 to 17 or a pharmaceutical composition according to Paragraph 18 for use in the treatment or prevention of rheumatoid diseases.

22. A compound or pharmaceutical composition for use according to Paragraph 20 or 21, wherein the disease is arthritis.

23. A compound or pharmaceutical composition for use according to any one of Paragraphs 20 to 22, wherein the disease is osteoarthritis.

24. A method of treating or preventing a disease characterised by hyaluronan overproduction comprising administering to a patient in need thereof a therapeutically effective amount of a compound as defined in any one of Paragraphs 1 to 17 or a pharmaceutical composition according to Paragraph 18.

25. A method of treating or preventing a rheumatoid disease comprising administering to a patient in need thereof a therapeutically effective amount of a compound as defined in any one of Paragraphs 1 to 17 or a pharmaceutical composition according to Paragraph 21.

26. A method of treatment according to Paragraph 24 or 25, wherein the disease is arthritis.

27. A method of treatment according to any one of Paragraphs 24 to 26, wherein the disease is osteoarthritis.

28. The use of a compound as defined in any one of Paragraphs 1 to 17 or a pharmaceutical composition according to Paragraph 18 for the manufacture of a medicament for the treatment of a disease characterised by hyaluronan overproduction.

29. The use of a compound as defined in any one of Paragraphs 1 to 17 or a pharmaceutical composition according to Paragraph 18 for the manufacture of a medicament for the treatment of a rheumatoid disease.

30. The use of a compound or pharmaceutical composition according to Paragraphs 28 or 29, wherein the disease is arthritis.

31. The use of a compound or pharmaceutical composition according to any one of Paragraphs 28 to 30, wherein the disease is osteoarthritis.

32. A compound of formula (I):

[hyaluronan production inhibitor]-[labile linker]-X (I)
or a pharmaceutically-acceptable salt thereof,
wherein the hyaluronan production inhibitor fragment is selected from the group consisting of Formula A, B, C, D, and E, wherein
formula A is:

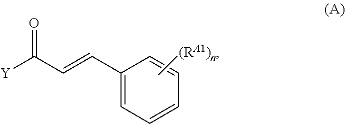

wherein Y is a group selected from the group consisting of:

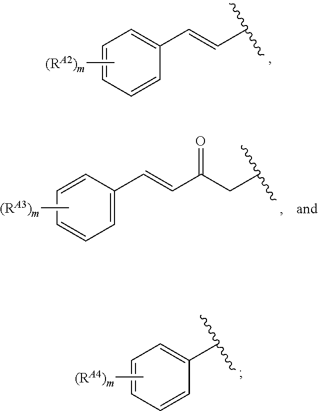

R^{A1}, R^{A2}, R^{A3}, and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2}, R^{A3} or R^{A4} group is

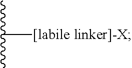

n is 0 to 5;
m is 0 to 5; and
R^{A5} is C₁₋₆ alkyl, C₂₋₆ alkenyl, C₂₋₆ alkynyl, which three groups are optionally substituted by one or more groups selected from halo, —OH and —OMe;
formula B is:

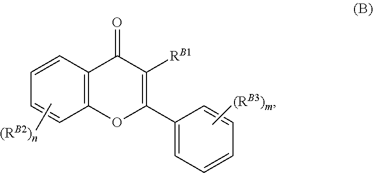

wherein R^{B1}, R^{B2}, and R^{B3} are each independently selected from the group consisting of —OH, —OR^{B4}, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe; and one R^{B2} or R^{B3} group is

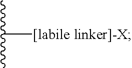

n is 0 to 4;
m is 0 to 5;
R^{B4} is C₁₋₆ alkyl, C₂₋₆ alkenyl, C₂₋₃ alkynyl, which three groups are optionally substituted by one or more groups selected from halo, —OH and —OMe;
formula C is:

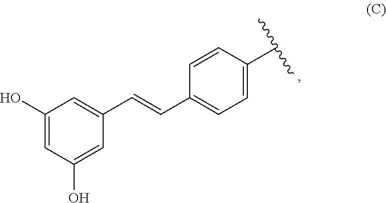 [SMILES: *c1ccc(/C=C/c2cc(O)cc(O)c2)cc1]

wherein the wavy line represents the point of attachment to the labile linker fragment;
formula D is:

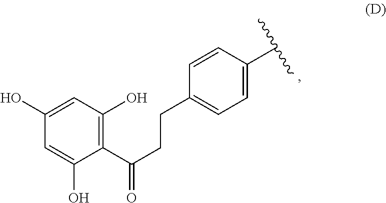 [SMILES: *c1ccc(CCC(=O)c2c(O)cc(O)cc2O)cc1]

wherein the wavy line represents the point of attachment to the labile linker fragment; or
formula E is:

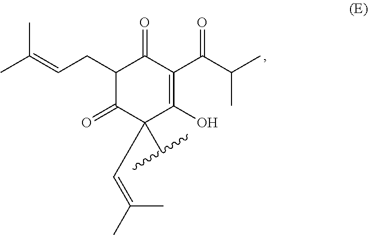 [SMILES: *C1(C=C(C)C)C(=O)C(CC=C(C)C)C(=O)C(C(=O)C(C)C)=C1O]

wherein the wavy line represents the point of attachment to the labile linker fragment;
the labile linker fragment is —OC(O)R^{L1}—;
wherein R^{L1} is selected from C₁₋₆ alkylene, C₂₋₆ alkenylene, C₂₋₆ alkynylene, arylene, or heteroarylene, which five groups are optionally substituted with one or more groups independently selected from halo, —OR^{L2} or ═O;
R^{L2} represents H or C₁₋₆ alkyl;
X is selected from the group consisting of quaternary ammonium, quaternary phosphonium, pyridinium and thiazolium salts; or

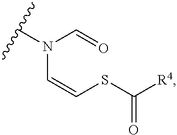

wherein R⁴ is C₁₋₆ alkyl,
for use in the treatment or prevention of a disease associated with hyaluronan overproduction, including rheumatoid diseases.

33. A compound for use according to Paragraph 32, or a pharmaceutically acceptable salt thereof, wherein the compound is of formula IV:

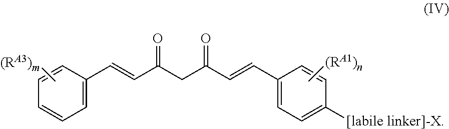

34. A compound for use according to Paragraph 32, or a pharmaceutically acceptable salt thereof, wherein the compound is of formula (V):

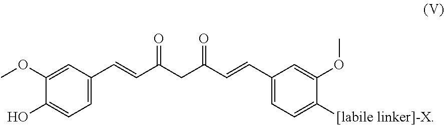

35. A compound for use according to Paragraphs 32 to 34, or a pharmaceutically acceptable salt thereof, wherein the use is for treatment or prevention of rheumatoid diseases.

36. A compound for use according to Paragraph 35, wherein the disease is arthritis.

37. A compound for use according to Paragraphs 32 to 36, wherein the disease is osteoarthritis.

38. A compound for use according to Paragraph 32, wherein the disease associated with hyaluronan overproduction is cancer.

39. A compound for use according to Paragraph 38, wherein the cancer is sarcoma, preferably chondrosarcoma.

40. A method of treating or preventing a disease characterised by hyaluronan overproduction comprising administering to a patient in need thereof a therapeutically effective amount of a compound as defined in any of Paragraphs 32 to 34.

41. A method of treating or preventing a rheumatoid disease comprising administering to a patient in need thereof a therapeutically effective amount of a compound as defined in any one of any of Paragraphs 32 to 34.

42. A method of treatment according to Paragraph 40 or 41, wherein the disease is arthritis, preferably osteoarthritis.

43. A method of treatment according to Paragraphs 40, wherein the disease is cancer, preferably sarcoma, more preferably chondrosarcoma.

44. The use of a compound as defined in any one of Paragraphs 32 to 34 for the manufacture of a medicament for the treatment of a disease characterised by hyaluronan overproduction.

45. The use of a compound as defined in any one of Paragraphs 32 to 34 for the manufacture of a medicament for the treatment of a rheumatoid disease.

46. The use of a compound according to Paragraphs 44 or 45, wherein the disease is arthritis, preferably osteoarthritis.

47. The use of a compound according to Paragraph 44, wherein the disease is cancer, preferably sarcoma, more preferably chondrosarcoma.

### EXAMPLES

The present invention will be further described by reference to the following examples, which are not intended to limit the scope of the invention.

### Abbreviations

The following abbreviations may be used herein.
DCM dichloromethane
DMSO dimethylsulfoxide
EtOAc ethyl acetate
LC-MS liquid chromatography-mass spectrometry
NMR nuclear magnetic resonance
THF tetrahydrofuran

### EXPERIMENTAL PROCEDURES

In the event that there is a discrepancy between nomenclature and any compounds depicted graphically, then it is the latter that presides (unless contradicted by any experimental details that may be given or unless it is clear from the context).

Starting materials and intermediates used in the synthesis of compounds described herein are commercially available or can be prepared by the methods described herein or by methods known in the art.

Experiments were generally carried out under inert atmosphere (nitrogen or argon), particularly in cases where oxygen- or moisture-sensitive reagents or intermediates were used.

Mass spectrometry data are reported from liquid chromatography-mass spectrometry (LC-MS) using electrospray ionization. Chemical shifts for NMR data are expressed in parts per million (ppm, δ) referenced to residual peaks from the deuterated solvent used.

For syntheses referencing general procedures, reaction conditions (such as length of reaction or temperature) may vary. In general, reactions were followed by thin layer chromatography or LC-MS, and subjected to work-up when appropriate. Purifications may vary between experiments: in general, solvents and the solvent ratios used for eluents/gradients were chosen to provide an appropriate R_{f} and/or retention time.

Where applicable, compound names indicated in respect of the following examples have been generated using the structure naming function of ChemBioDraw Ultra, Version 12.0.

### Example Compounds

### Example 1: 2-(4-((1E,4E)-5-(4-hydroxy-3-m ethoxyphenyl)-3-oxopenta-1,4-dien-1-yl)-2-methoxyphenoxy)-N,N,N-trimethyl-2-oxoethanaminium chloride (Hylin-betaine)

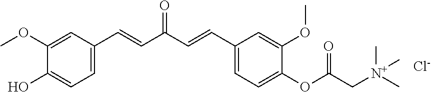

### 1,5-Bis(4hydroxy-3-methoxyhenyl)-1,4-pentadien-3-one (Hylin)

Hylin was synthesised according to a published procedure (Quincoces Suarez J. A. et al., 15 Bioorg Med Chem. 2010, 18, 6275-81).

Vanillin (0.2 mol; 30.4 g) and acetone (0.1 mol; 7.3 ml) were mixed and subjected to ultrasonication at 40° C. in the presence of concentrated hydrochloric acid (2 ml) for 2 hours. The mixture was poured into an ice-water mixture. The crude product was dissolved in a sodium hydroxide solution, and the filtrate was neutralized with hydrochloric acid. The precipitate was filtered, washed with water and dried to obtain hylin as a yellow powder (Yield: 80%). ¹H NMR (DMSO-d₆): δ=3.65 (6H, s), 6.55 (2H, d, J=15.7 Hz), 6.78 (2H, dd, J=8.4, 0.4 Hz), 7.23 (2H, dd, J=1.9, 0.4 Hz), 7.38 (2H, d, J=15.7 Hz), 7.58 (2H, dd, J=8.4, 1.9 Hz).

### 4-[(1E,4E)-5-(4-hydroxy-3-methoxyphenyl)-3-oxopenta-1,4-dien-1-yl]-2-methoxyphenyl chloroacetate

Hylin (30 g; 93 mmol) and pyridine (9.7 ml; 120 mmol) were dissolved in 120 ml of THF and heated to boiling. Chloroacetylchloride (9.6 ml; 120 mmol) was added dropwise to the boiling solution. The solution was refluxed for an additional hour and after cooling added to a mixture of water and DCM. The organic layer was washed with 0.5 N HCl, evaporated and dried over MgSO₄. The product was purified on a column of silica gel with DCM/MeOH (100:2) as eluent (Yield: 54%). ¹H NMR (DMSO-d₆): δ=3.76 (3H, s), 3.78 (3H, s), 4.21 (2H, s), 6.57 (1H, d, J=15.6 Hz), 6.67 (1H, d, J=15.7 Hz), 6.78 (1H, dd, J=8.4, 0.4 Hz), 7.21-7.27 (2H, 7.24 (dd, J=8.3, 0.5 Hz), 7.23 (dd, J=1.9, 0.4 Hz)), 7.34-7.43 (2H, 7.41 (dd, J=1.9, 0.5 Hz), 7.39 (d, J=15.6 Hz)), 7.49-7.61 (2H, 7.58 (dd, J=8.4, 1.9 Hz), 7.54 (d, J=15.7 Hz)), 7.73 (1H, dd, J=8.3, 1.9 Hz).

### 2-(4-((1E,4E)-5-(4-hydroxy-3-methoxyphenyl-3-oxopenta-1,4-dien-1-yl)-2-methoxyphenoxy)-N,N,N-trimethyl-2-oxoethanaminium chloride (Hylin-betaine)

4-[(1 E,4E)-5-(4-hydroxy-3-methoxyphenyl)-3-oxopenta-1,4-dien-1-yl]-2-methoxyphenyl chloroacetate (18.8 g; 50 mmol) was dissolved in 100 ml of THF and a solution of 2 M trimethylamine in THF (120 ml; 240 mmol) was added. The solution was stirred overnight at room temperature. The precipitate was filtered, washed extensively with DCM and dried in vacuum (Yield: 60%). ¹H NMR (DMSO-d₆): δ=2.88 (9H, s), 3.75 (3H, s), 3.78 (3H, s), 4.06 (2H, s), 6.57 (1H, d, J=15.6 Hz), 6.67 (1H, d, J=15.7 Hz), 6.78 (1H, dd, J=8.4, 0.4 Hz), 7.22-7.28 (2H, 7.23 (dd, J=1.9, 0.4 Hz), 7.25 (dd, J=8.3, 0.5 Hz)), 7.34-7.43 (2H, 7.39 (d, J=15.6 Hz), 7.41 (dd, J=1.9, 0.5 Hz)), 7.49-7.61 (2H), 7.54 (d, J=15.7 Hz), 7.58 (dd, J=8.4, 1.9 Hz)), 7.73 (1H, dd, J=8.3, 1.9 Hz).

### Example 2: (E)-2-(4-(3-(2,4-dihydroxy-6-methoxy-3-(3-methylbut-2-en-1-yl)phenyl)-3-oxoprop-1-en-1-yl)phenoxy)-N,N,N-trimethyl-2-oxoethanaminium chloride (Xantho-betaine)

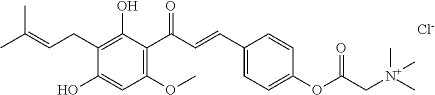 [SMILES: COc1cc(O)c(CC=C(C)C)c(O)c1C(=O)/C=C/c1ccc(OC(=O)C[N+](C)(C)C)cc1 ; [Cl-]]

Betaine (0.32 g, 2.7 mmol) was converted to the acylchloride by heating with 3 ml of thionylchloride at 80° C. for 30 min. Excess thionylchloride was evaporated and residual traces were co-evaporated with toluene. The betainchloride was dissolved in 5 ml of dry pyridine and added to 0.34 g of xanthohumol. After stirring for 1 hour, solvent was evaporated and residual traces were again co-evaporated with toluene. The residue was dissolved in 3 ml of methanol and xanthohumol-betaine ester was precipitated by addition of 4 ml tetrahydrofuran. ¹H NMR (DMSO-d₆): δ=1.66-1.67 (6H, 1.66 (s), 1.66 (s)), 2.89 (9H, s), 3.22 (2H, d, J=6.7 Hz), 3.95 (3H, s), 4.06 (2H, s), 5.31 (1H, t, J=6.7 Hz), 6.15 (1H, s), 6.70 (1H, d, J=15.6 Hz), 7.35 (2H, ddd, J=8.2, 1.4, 0.5 Hz), 7.40-7.50 (3H, 7.47 (ddd, J=8.2, 1.5, 0.5 Hz), 7.44 (d, J=15.6 Hz)).

### Example 3: 4-((E)-3-(2,4-dihydroxy-6-methoxy-3-(3-methylbut-2-en-1-yl)phenyl)-3-oxoprop-1-en-1-yl)phenyl 2-(N-((Z)-2-(acetylthio)vinyl)formamido)acetate (Xantho-prothiazole)

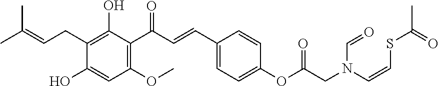 [SMILES: COc1cc(O)c(CC=C(C)C)c(O)c1C(=O)/C=C/c1ccc(OC(=O)CN(C=O)/C=C\SC(C)=O)cc1]

### 5-N-4-acetic acid)-thiazolonium iodide

Thiazole (20 g, 580 mMol) was refluxed with iodoacetic acid (120 g, 580 mMol) in 300 ml of dichloromethane for 2 days. After cooling the room temperature, the precipitate was isolated by filtration (101 g; 64%).

### {[2-(acetylthio)vinyl](formyl)amino}acetic acid

The thiazolium ring was opened and the thiole was acetylated as described by Koltunova and Berezovskii (Koltunova, V. I. and Berezovskii, N. M. (1969) Coenzymes and phosphates. XXI. S-acyl and N,Sdiacylcocarboxylase and other N,S-derivatives of thiamine phosphates. The Journal of General Chemistry of the USSR 39, 102-109).

Briefly, 5-N-(4-acetic acid)-thiazolonium iodide (20 g; 73.8 mMol) was mixed with 10 ml of water and dissolved by slow addition of 10 M NaOH. The reaction vessel was cooled in an ice bath and 10 M NaOH was added, while the pH was maintained between 10 and 11. The thiazole rings opened and required further addition of NaOH in 10 min intervals over a period of 3 hours. Acetylchloride (10.8 ml; 150 mMol) was added dropwise, while the pH was maintained between 10 and 11 with 10 M NaOH and the temperature below 15° C. The solution was brought to pH 3.6 with concentrated HCl and the precipitated product was isolated by filtration (6.3 g; 42%).

### Xanthohurmol-2-{[2-(acetylthio)vinly]formyl)amino}acetate ester

Compound {[2-(acetylthio)vinyl](formyl)amino}acetic acid was dissolved in 5 ml of tetrahydrofuran and esterified with xanthohumol by the Steglich reaction. 4-Dimethylaminopyridine (0.06 g; 0.5 mMol) and alkali treated xanthohumol (0.89 g, 2.5 mMol) were added. The solution was cooled to 0° C. and dicyclohexylcarbodiimide (0.52 g, 2.5 mMol) was added. After stirring over night at room temperature, the precipitated was removed by filtration and the solution was concentrated by evaporated. The xanthohumol-prothiazole ester was purified by preparative thick layer chromatography with chloroform/methanol (50:3). Yield 20%.

¹H NMR (DMSO-d₆): δ=1.56-1.57 (6H, 1.56 (s), 1.56 (s)), 2.11 (3H, s), 3.22 (2H, d, J=6.7 Hz), 3.95 (3H, s), 4.07 (2H, s), 5.31 (1H, t, J=6.7 Hz), 5.52 (1H, d, J=9.9 Hz), 6.15 (1H, s), 6.70 (1H, d, J=15.6 Hz), 6.91 (1H, d, J=9.9 Hz), 7.36 (2H, ddd, J=8.3, 1.4, 0.5 Hz), 7.40-7.50 (3H, 7.47 (ddd, J=8.3, 1.5, 0.5 Hz), 7.44 (d, J=15.6 Hz)), 8.61 (1H, s).

### Example 4: (Z)-2-hydroxy-4-(3,5,7-trihydroxy-4-oxo-4H-chromen-2-yl)phenyl 2-(N-(2-(acetylthio)vinyl)formamido)acetate (Quercetin-prothiazole)

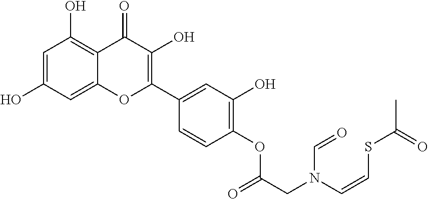 [SMILES: CC(=O)S/C=C\N(C=O)CC(=O)Oc1ccc(-c2oc3cc(O)cc(O)c3c(=O)c2O)cc1O]

Quercetin-prothiazole was obtained in a similar reaction as shown in Example 3, using 0.76 g of quercetin.

¹H NMR (DMSO-d₆): δ=2.11 (3H, s), 5.95 (1H, d, J=9.8 Hz), 6.27 (1H, d, J=1.9 Hz), 6.44 (1H, d, J=1.9 Hz), 7.12 (1H, d, J=9.8 Hz), 7.41 (1H, dd, J=8.4, 0.5 Hz), 7.45 (1H, dd, J=1.8, 0.5 Hz), 7.79 (1H, dd, J=8.4, 1.8 Hz), 8.74 (1H, s).

### Biological Assays

The biological activity of example compounds as described herein above was assessed using the following biological assays.

### General Methods

The hyaluronan synthase activity (Prehm, P. and Schumacher, U., Biochem. Pharmacol. 2004, 68, 1401-1410), the cytotoxicity of the drugs (O'Brien, J. et al., Eur. J. Biochem. 2000, 267, 5421-5426), proteoglycan synthesis rate, hyaluronan (Prehm, P. J. Rheumatol., 2005, 32, 690-696) and proteoglycan (Bjornsson, S. Anal. Biochem. 1993, 210, 282-291) were determined as described. The effect of the prenylflavonoids on the MRP5 export rate was measured with fluorescein diacetate as described (Schulz, T., Schumacher, U., and Prehm, P., J Biol. Chem., 2007, 282, 20999-21004). The determination of the IC50 values on the phosphodiesterase 5 was performed according to the instructions of BPS Bioscience. The culture of bovine chondrocytes in alginate beads and of explants of bovine cartilage was described previously (Deiters, B. and Prehm, P. Arthritis Res. Ther., 2008, 10, R8; Prehm, P. J. Rheumatol. 2005, 32, 690-696). Osteoarthritic reactions were induced in chondrocytes growing in alginate beads by incubation with medium containing 10 ng/ml of II-17 for 6 days, and in cartilage explants with 10 ng/ml of II-17 and 5 ng/ml of II-1α. The supernatants were withdrawn and stored at −20° C. for later analysis. The explants were weighed and extracted with 1 ml of 4 M guanidinium-HCl for 72 hours at 4° C. The proteoglycan concentrations were determined in the extracts.

### Materials

Bovine articular cartilage was from a local slaughterhouse. HABP was from Calbiochem, hyaluronan (Healon®) was a gift from Genzyme, Cambridge, MA. Xanthohumol was a gift from Dr. Martin Biendl, Hallertauer Hopfenveredelungsgesellschaft, Mainburg, Germany. Additional chemicals were from Sigma-Aldrich Chemical Corporation.

### Cell Culture

Chondrosarcoma cells (SW1353 from the American Type Culture Collection) were grown in Dulbecco's medium supplemented with streptomycin/penicillin (100 units of each/ml), 10% foetal calf serum or in serum free Quantum medium supplemented with streptomycin/penicillin (100 units of each/ml), kanamycin (100 units/ml).

### Histochemical Staining of Collagen in Cartilage

Cartilage explants were incubated with and without a mixture of 10 ng/ml of II-1α, 10 ng/ml of II-1β and 25 ng/ml of II-17 for 14 days. The addition of the cytokines was repeated daily. The tissues were fixed with 3.7% paraformaldehyde for 24 hours, imbedded in paraffin and stained by the van Giesson method.

### Zymography of Matrix Proteases

Bovine chondrocytes in alginate beads were cultured in serum free DMEM for 3 days in the absence or presence of 11-17 (25 ng/ml) and 12.5 μM or 50 μM of the inhibitors xanthohumol, isoxanthohumol, 8-prenylnaringenin and icariin. The protein concentrations of the culture media were determined and equal amounts of proteins were directly applied to a 7.5% SDSpolyacrylamide gel that contained 0.1% gelatin. After electrophoresis, the gel was washed twice with 2.5% Triton X-100 for 30 min, three times with water for 10 min and a solution of 50 mM Tris-HCl, 5 mM CaCl₂), 1 μM ZnSO4, pH 8.0 for 5 days at 37° C. The gel was stained with Coomassie blue.

### Determination of Proteoglycans

Cartilage explants were weighed (average wet weight: 20 mg) incubated in the absence and presence of IL-1 (2 ng/ml) and the inhibitors at various concentrations for 5 days. The tissues were extracted with 1.5 ml of a solution of 4 M guanidinium hydrochloride, 0.1 M ε-aminohexanoid acid, 5 mM benzamidine, 10 mM N-ethylmaleinimide, 0.5 mM phenylmethylsulfonyl fluoride for 3 days at 4° C. The solution was centrifuged for 5 min at 10.000 g and the proteoglycans were determined in the supernatant by the alcian blue method as described (Bjornsson, S., Anal. Biochem., 1993, 210, 282-291).

### Histochemical Staining of Collagen in Cartilage

Cartilage explants were incubated with and without a mixture of 10 ng/ml of 11-17 and 5 ng/ml of II-1α for 14 days. The addition of the cytokines was repeated daily. The tissues were fixed with 3.7% paraformaldehyde for 24 hours, imbedded in paraffin and stained by the van Gieson method (van Gieson, 1. (1889) Laboratory notes of technical methods for the nervous system. New York Medical Journal 50, 57-60).

Uptake of Flavonoids into Chrondrosarcoma Cells

Confluent monolayers of chondrosarcoma cells on 96 well microtiter plates were incubated with 100 μM solutions of flavonoids in serum-free Quantum medium. Changes in fluorescence were monitored from the bottom of the wells at excitation and emission wavelengths of 340 nm and 450 nm for xanthohumol and at 440 nm and 530 nm for quercetin, respectively.

### Results

### Example 5: Toxicity

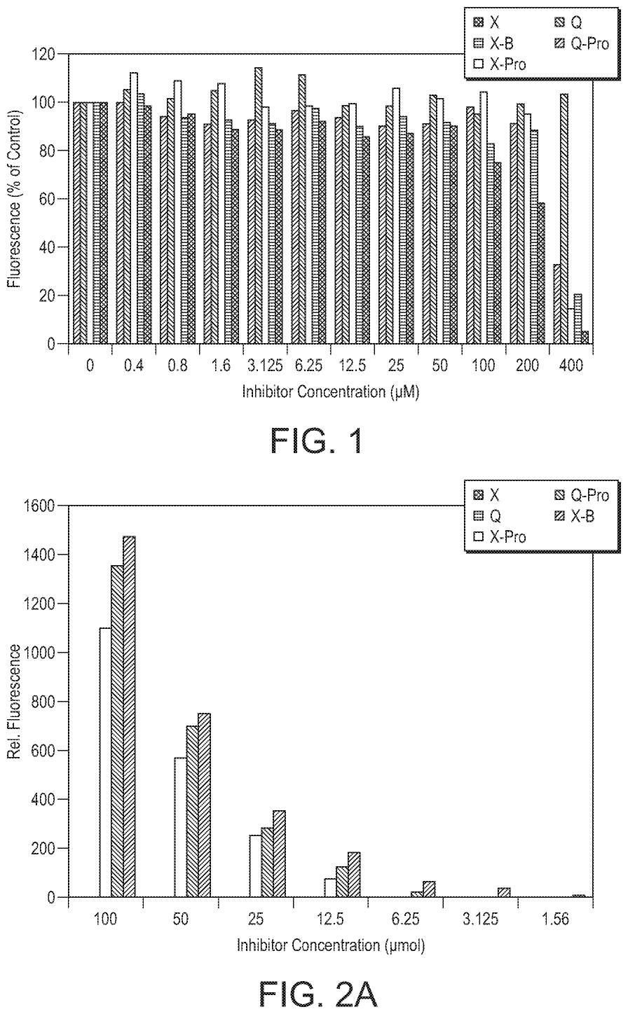

The toxicity was determined by the Alamar blue assay. FIG. 1 shows that all of the tested compounds were not toxic below concentrations of 100 μM.

### Example 6: Uptake of Flavonoids into Chondrosarcoma Cells

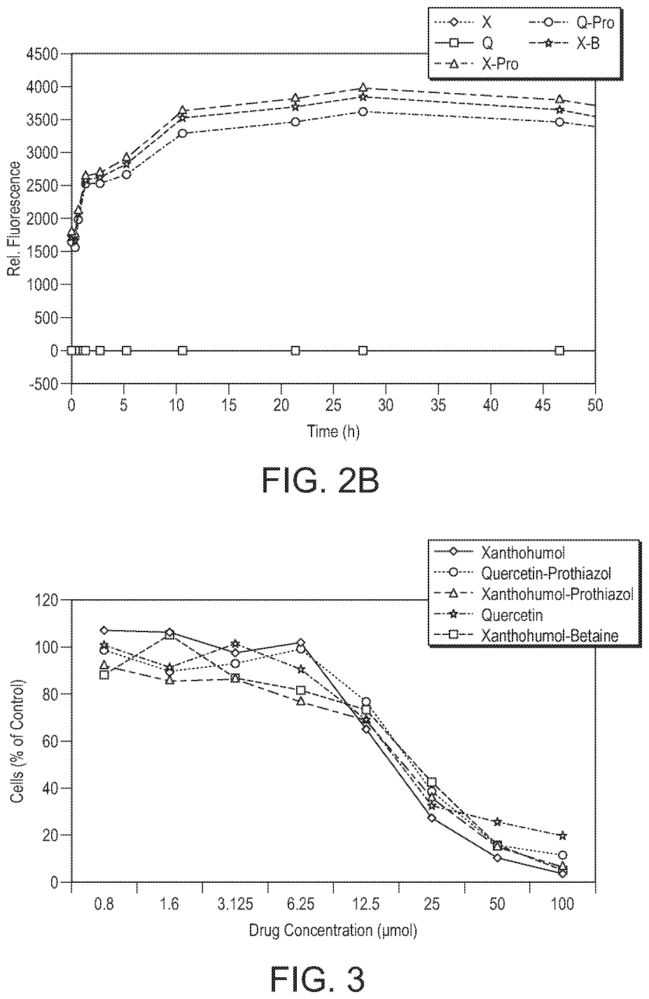

Flavonoids are weakly fluorescent in aqueous buffer, but greatly enhance the fluorescence in the presence of proteins (Sengupta, B. and Sengupta, P. K., Biochem. Biophys. Res. Commun., 2002, 299, 400-403). This property was used to follow the uptake of quercetin and xanthohumol into chondrosarcoma cells. FIG. 2A shows that the derivatised flavonoids were taken up in a concentration dependent manner, whereas the original xanthohumol and quercetin did not accumulate inside the cells. FIG. 2B shows that the uptake was initially very rapid and reached a plateau after 10 hours.

### Example 7: Inhibition of Cell Growth

Chondrosarcoma cells were plated on a 24 well microtiter plate and in the presence of increasing flavonoid concentrations. After 2 days the cells were trypsinized and the cell numbers were determined in a Coulter counter.

It is known that hyaluronan synthesis is required for cell mitosis and growth of fibroblasts and melanoma cells. Therefore we tested the effect of flavonoids on the growth of chondrosarcoma cells. FIG. 3 shows that the flavonoids inhibited cell growth in a concentration dependent manner. The prothiazole derivatives of xanthohumol and quercetin (i.e. Compounds of Examples 3 and 4) were more effective at submicromolar concentrations.

### Example 8: Inhibition of Hyaluronan Export

Chondrosarcoma cells were plated on a 96 well microtiter plate and in the presence of increasing flavonoid concentrations. After 2 days the concentration of hyaluronan was determined in the culture supernatant.

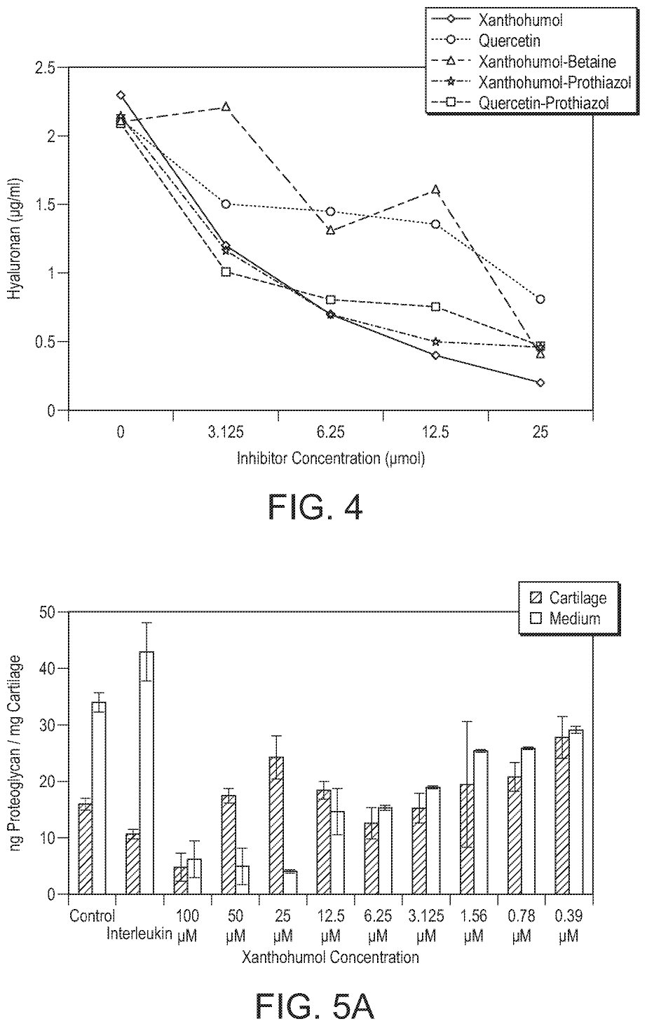

The flavonoids and their derivatives were analysed for their effects on hyaluronan export from chondrosarcoma cells. FIG. 4 shows that all compounds inhibited hyaluronan export in a concentration dependent manner. Xanthohumol-betaine (Compound of Example 2) was slightly less effective than xanthohumol, whereas xanthohumol-prothiazole (Compound of Example 3) and quercetin-prothiazole (Compound of Example 4) were more effective than their parent compound particularly at concentrations below 3 μM.

### Example 9: Inhibition of Proteoglycan Shedding from Bovine Cartilage Slices

Hyaluronan overproduction causes massive shedding of proteoglycans from cartilage. Bovine cartilage slices were cultivated in organ culture and osteoarthritic reactions were induced by addition on Interleukin and increasing concentrations of xanthohumol-betaine.

The amounts of proteoglycan were determined in the cartilage slices and in the medium after one week of incubation.

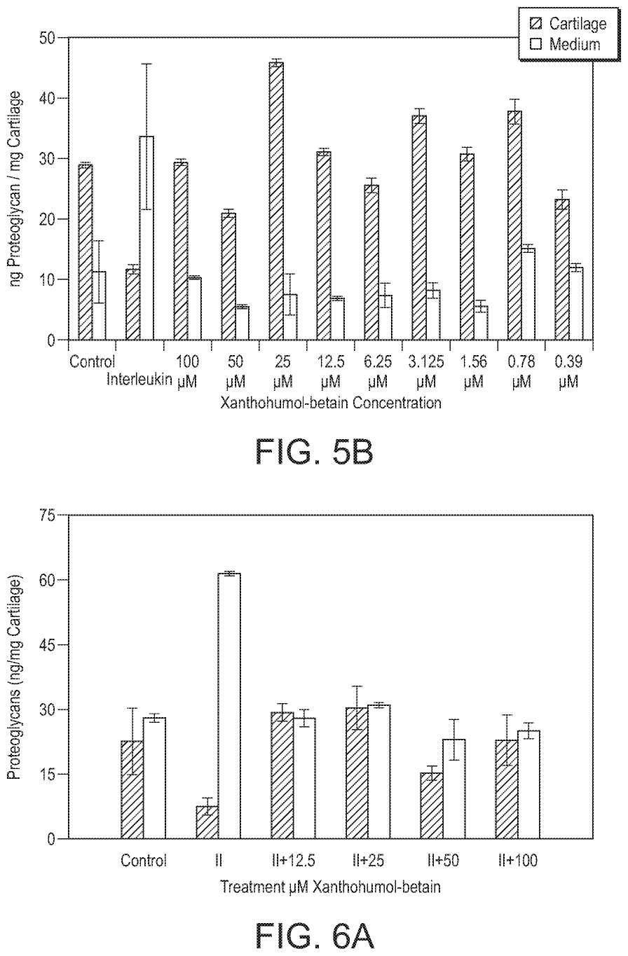

Compared to the unmodified xanthohumol (FIG. 5A), xanthohumol-betaine (FIG. 5B) appeared to be more effective at reducing shedding of proteoglycan into the medium.

### Example 10: Inhibition of Proteoglycan Loss

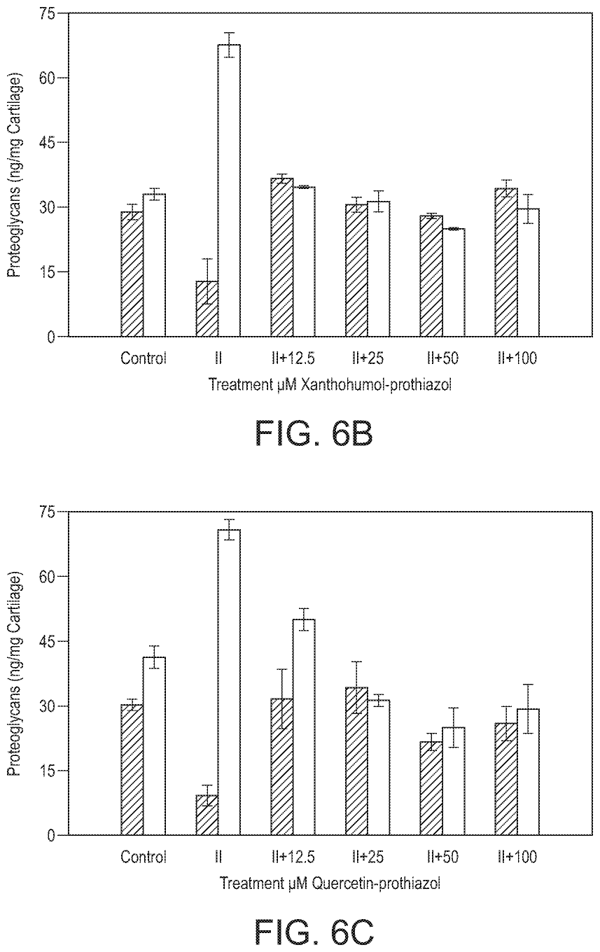

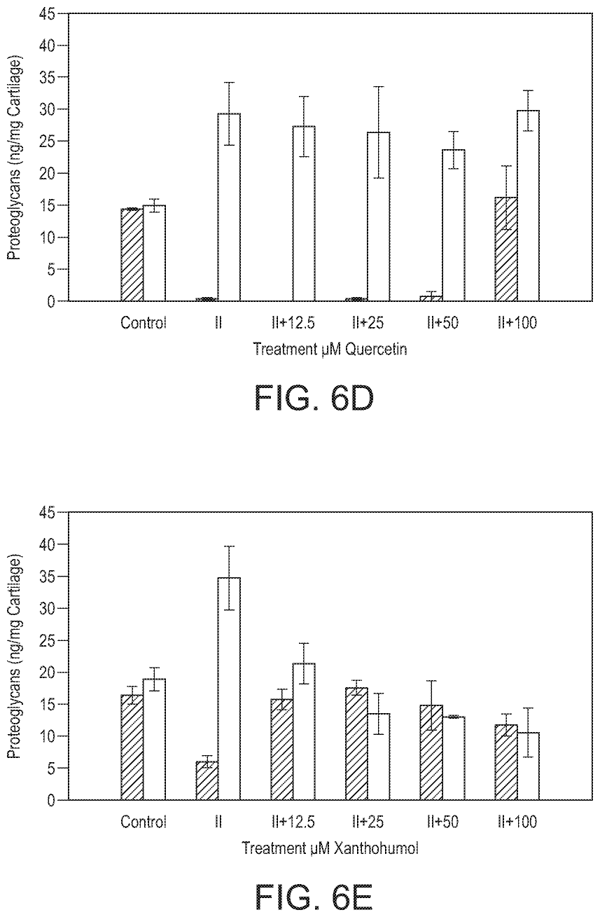

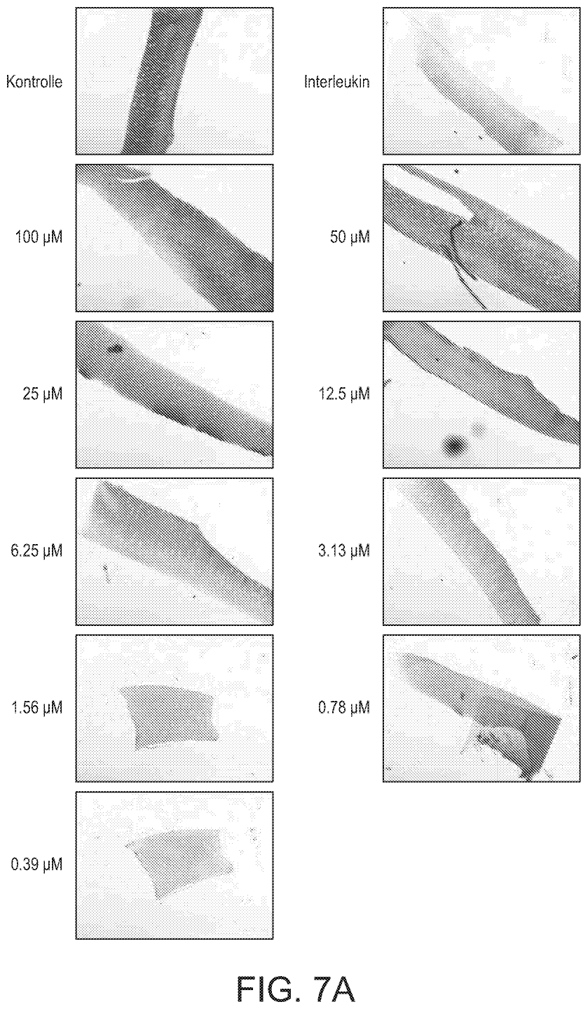

Bovine cartilage explants were incubated in the absence and presence of interleukin for stimulation of osteoarthritic reactions and the indicated concentrations of the xanthohumol-betaine (FIG. 6A), xanthohumol-prothiazole (FIG. 6B), quercetin-prothiazole (FIG. 6C), quercetin (FIG. 6D) and xanthohumol (FIG. 6E) for 5 days. The cartilage explants were weighed and the proteoglycan concentrations were determined in the cartilage explants (solid bars) and culture supernatants (open bars).

It has previously been reported that prenylflavonoids also inhibited loss of proteoglycans from interleukin activated bovine cartilage explants (Stracke, D., et al., Mol. Nutr. Food Res., 2011, 55, 485-494). Here the flavonoid-derivatives were analysed for their protective effects. FIG. 6 shows that xanthohumol-betaine (Compound of Example 2), xanthohumol-prothiazole (Compound of Example 3) and quercetin-prothiazole (Compound of Example 4) were similarly effective.

### Example 11: The Prenylflavonoids Reduce Collagen Degradation

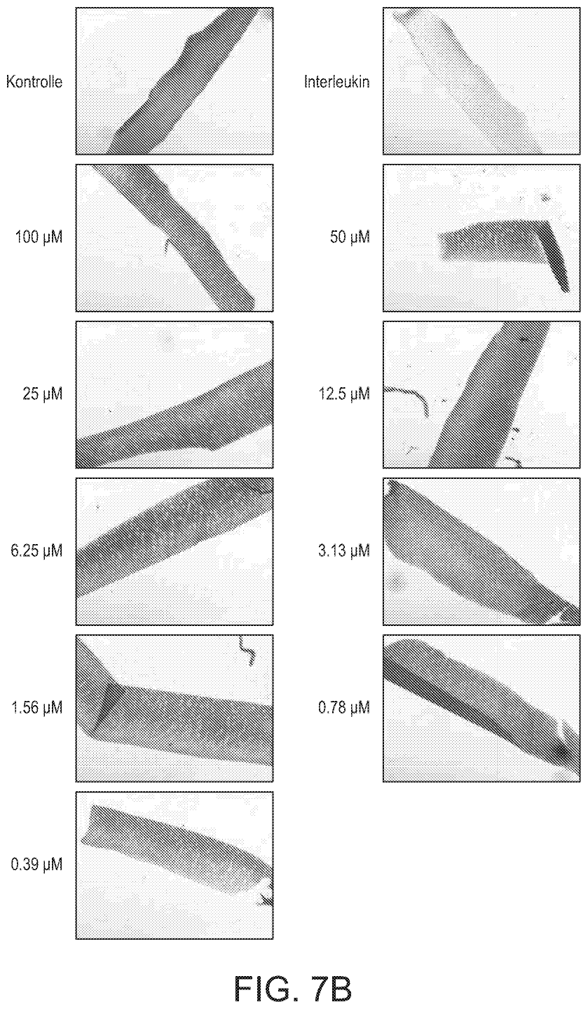

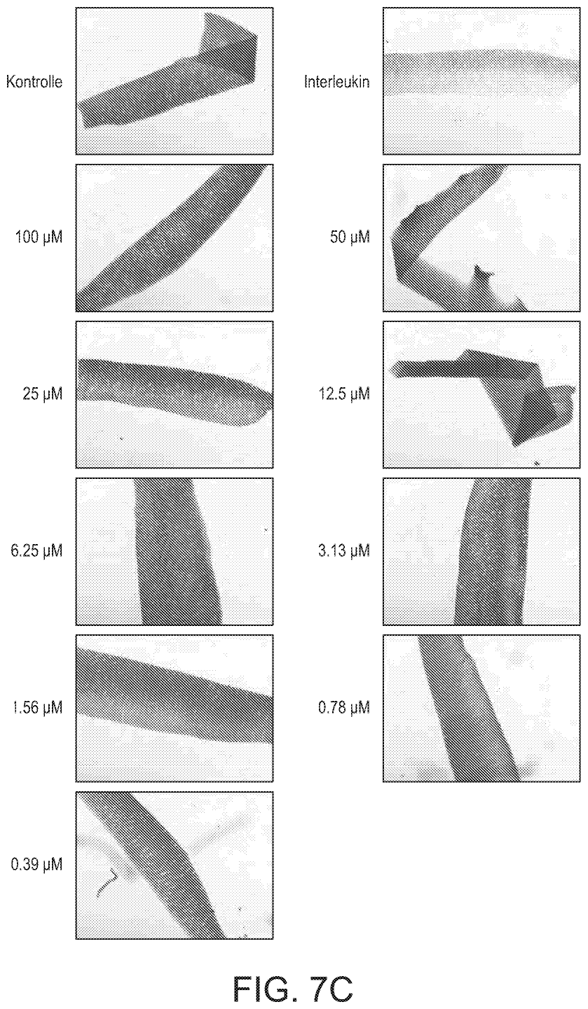

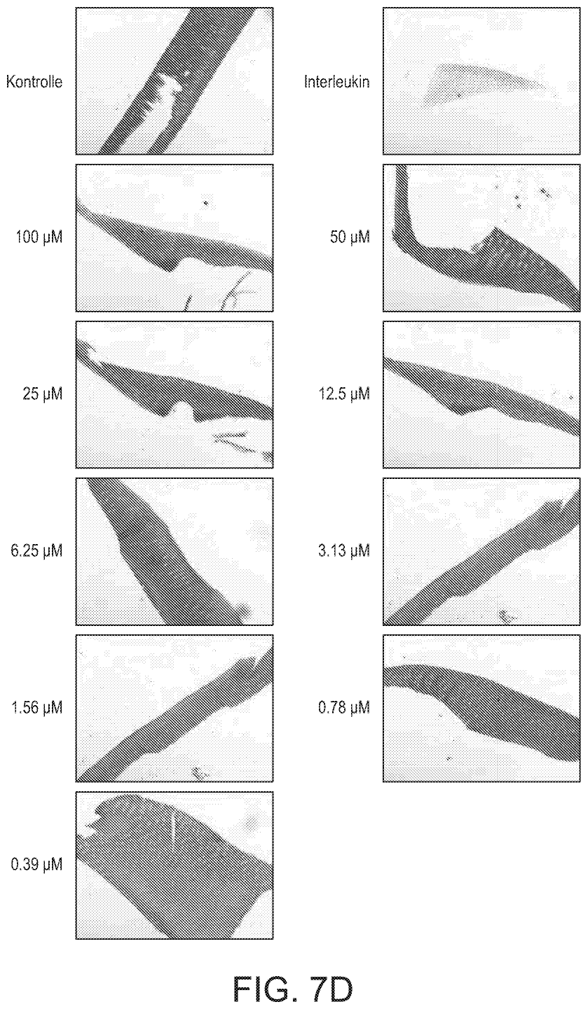

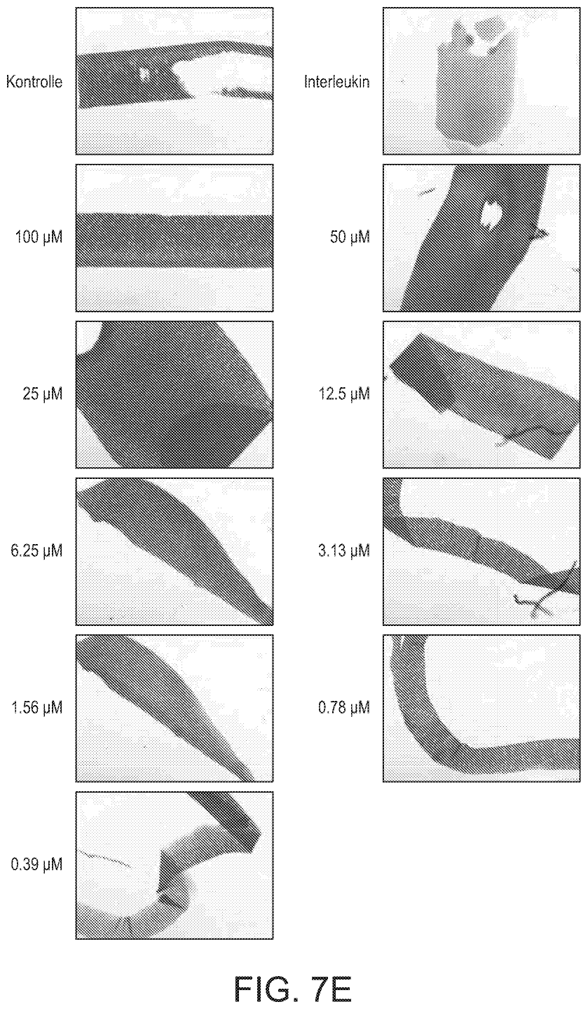

It is known that hyaluronan overproduction leads to collagen degradation from cartilage (Deiters, B. and Prehm, P., Arthritis Res. Ther., 2008, 10, R8; Prehm, P., J. Rheumatol., 2005, 32, 690-696). Initial experiments with II-17 alone indicated that this treatment was insufficient to induce detectable collagen degradation. Therefore cartilage explants were incubated with a mixture of the osteoarthritis inducing chemokines II-1α, II-1β and II-17 with 12.5 μM and 50 μM of the prenylflavonoids for 14 days and collagen was stained by the van Giesson method. FIGS. 7A to 7E show that particularly xanthohumol was effective at protecting the cartilage from collagen loss.

### Example 12: IC50s

TABLE 1
IC50-values of test compounds obtained from three assays (in μmol).

| | Alamar blue | Alamar blue | Inhibition of | Inhibition of |
|---|---|---|---|---|
| | staining of | staining of | proteoglycan | hyaluronan |
| Compound | fibroblasts | chondrosarcoma | loss | production |
| Xanthohumol | 55 | 76 | 2.6 | 19 |
| Xathohumol- | 80 | 275 | 0.3 | 18 |
| betaine | | | | |
| Xanthohumol- | 74 | 69 | — | 20 |
| Prothiazole | | | | |
| Quercetin | — | 70 | — | 16 |
| Quercetin- | — | — | — | — |
| Betaine | | | | |
| Quercetin- | 66 | 61 | — | 8 |
| Prothiazole | | | | |

### In Vivo Studies

The experiments described below explore whether xantho-betaine and hylin-betaine treatments modify gait, bone architecture and slows/reverses articular cartilage destruction in a spontaneous osteoarthritis (OA) model in Str/ort mice.

### Animals

The Str/ort mice were housed in a pathogen free facility at 21±2° C. with 12-hour light/dark cycles at the Royal Veterinary College (RVC) in polycarbonate or polypropylene cages with wood chip and paper bedding. The mice were housed up to 4 per a cage; weaners up to 8 weeks of age were fed a standard rodent breeding diet and thereafter a standard rodent maintenance diet (Special Diet Services, South Witham, UK). All of the procedures conducted in the facility were in accordance with the Animals Act (Scientific Procedures) 1986 and local RVC ethical guidelines.

### “Hylin-Betaine” and “Xantho-Betaine” Treatment

At 10-13 weeks of age, 21 male Str/ort mice (n=7/group) were assigned randomly to vehicle, hylin-betaine and xantho-betaine groups and were treated intraperitoneally 18 weeks with either vehicle (5% DMSO in 95% peanut oil), 50 mg/kg compound of Example 1 (“hylin-betaine”) in 5% DMSO and 95% peanut oil or 50 mg/kg compound of Example 2 (“xantho-betaine”) in 5% DMSO and 95% peanut oil.

### Health Evaluation

To assess the health status after hylin-betaine and xantho-betaine treatment, mice were monitored for their health status. The monitoring criteria were body surface temperature, body weight, gait, coat condition, skin tenting for dehydration, hunching, activity, breathing rate and general body condition. Mice were scored blind for each one of these criteria from 1-5 except for body weight and temperature.

### Gait Analysis

Gait was monitored using a treadmill-based video system (DigigaitTM system) in three treatment groups of mice at three time point, week 1, week 9 and week 18 of treatment. The number of mice was 7, 7 and 5 in groups 1, 2 and 3, respectively.

Two hundred and seventy-nine gait descriptors were used for the analyses. Thirty-four descriptors were obtained directly from the software of the system in the 4 limbs (LF: left front; RF: right front; LR: left rear; RR: right rear). All the others (143) were calculated indexes from gait measurements including swing, stance, brake, propel and stride times, stride length, frequency and coefficient of variation, paw area and paw angle and the variability of those two traits, the coefficient of variation of swing duration mid line distance, and minimum and maximum dAdT. The ratios of times were calculated in the 4 limbs: Brake, Propel, and Swing times relative to Stance and Stride times, Stance/Swing and Stance/Stride. In addition, the following additional indexes were considered:

Symmetry index(SI; 100*(|R−L|)/[0.5*(R+L)]

### Symmetry Ratios:

¾ Minimum of the ratios between Hind and Fore Limbs in the same (right and left) side
¾ Minimum of the ratios between both fore limbs and between both hind limbs

### Compensation or Balance Between the Contralateral Fore and the Hind Limbs:

¾ Between the right-hind and left fore-limbs: dgr=|RR−LF|
¾ Between the left-hind and right fore-limbs: dgl=|LR−RF|
where RR, LR, RF, LF referred to right-rear, left-rear, right-fore and left-fore limbs. The minimum and the maximum of those values, as well as the minimum of the ratio between them were computed.

These measures of asymmetry allow for the unpredictable targeting of OA to specific hind-limb knee joints in this strain of Str/ort mouse. Greater symmetry indicates more ‘normal’ gait pattern (with the proviso that alternatively both limbs may be equally affected).

In order to investigate whether there were differences among groups for each descriptor at the three experimental dates, an ANOVA was performed using data corresponding to each descriptor and date. Bonferroni correction was applied to counteract the problem of multiple comparisons.

A t-test analysis was also performed comparing only two groups at a time. In order to assess whether differences between both groups could be due to differences in gait change regarding the initial condition (at 2014/12/23), two sets of analyses were performed for data at 2015/02/18 and 2015/04/14, respectively by using Logistic Regression Analysis. The model included two independent variables: the initial value for each descriptor and the difference between the corresponding value at 2015/02/18 (or 2015/04/14) and the initial value. Bonferroni correction was also applied.

### High-Resolution Micro-Computed Tomoaraphy (Micro-CT)

### I. Scanning

Micro-CT scanning and analysis were performed as described previously. Briefly, tibiae from vehicle, hylin-betaine and xantho-betaine treated groups were scanned using the Skyscan 1172 (Skyscan, Kontich, Belgium), with x-ray tube operated at 50 kV and 200 Micro-A, 1600 ms exposure time with a 0.5 mm aluminium filter and a voxel size of micro-m 5. The scanning time for each sample was approximately 2 hours. The slices were then reconstructed using NRecon 1.6.9.4 (Skyscan, Kontich, Belgium). 2D/3D analyses were performed using CTAn 1.15.4.0+ version software (Skyscan, Kontich, Belgium). Additionally, 3D visualization and production of colour-coded images of trabecular, cortical and skull bones were conducted using Avizo 9.0.0 software (FEI, Oregon USA).

### II. Morphormetrical Analysis

a. Trabecular analysis: prior to analysis, Micro-CT images were re-oriented in DataViewer 1.5.0 (Skyscan, Kontich, Belgium), such that the cross-section within the transverse plane was perpendicular to the long axis of the bone. Tibial length was measured in CTAn 1.15.4.0+ software using a straight line measuring tool and the appearance of the trabecular ‘bridge’ connecting the two primary spongiosa bone ‘islands’ was set as reference point for analysis of the metaphyseal trabecular bone adjacent to the epiphyseal growth plate. 5% of the total bone length from this point (towards the diaphysis) was utilised for trabecular analysis of the proximal tibia. The trabecular region of interest was drawn freehand using the selection tool of CTAn, a few voxels away from the endocortical surfaces in order to avoid inclusion of remnants of primary spongiosa associated with cortical bone. The selected trabecular regions of interests were analysed using CTAn BatMan software (Skyscan, Kontich, Belgium) and morphometric parameters were recorded.
b. Whole bone cortical analysis: Whole bone analysis was performed on datasets derived from CT scans using BoneJ (version 1.4.0), an ImageJ plugin. Following segmentation, alignment and removal of fibula from the dataset, a minimum bone threshold was selected using a histogram based method in ImageJ which utilises all pixels in a stack to construct a histogram and was further confirmed using ImageJ “threshold function”. The threshold ranged between 22000-22100 and was applied to all datasets to separate higher density bone from soft tissues and air. This threshold was used in “Slice Geometry” function within BoneJ to calculate bone cross sectional area (CSA), second moment of area around the minor axis (Imin), second moment of area around the major axis (Imax), mean thickness determined by local thickness in two dimensions (Ct.Th), ellipticity and resistance to torsion (J). The most proximal (15%) and the most distal portions (25%) of tibial length were excluded from analysis, as these regions include trabecular bone.

### Histology and Grading of Articular Cartilage (AC) Lesions

Dissected right knees were fixed in neutral buffered formalin, decalcified (Immunocal, Quartett, Berlin), wax-embedded and 6 μm coronal sections cut. Multiple sections (five/slide) from 120 μm intervals across the whole joint were stained with Tollidine blue (0.1% in 0.1 M acetate buffer, pH 5.6) and AC lesion severity graded.

Sections from right vehicle, hylin-betaine and xantho-betaine treated Str/ort joints were stained with Safranin O. Sections were dewaxed, and AC lesion severity was scored in Str/ort mice by the methods of Chambers et al., (Chambers et al Expression of collagen and aggrecan genes in normal and osteoarthritic murine knee joints. Osteoarthritis and cartilage/OARS, Osteoarthritis Research Society. 2002; 10(1):51-61) consistent with an internationally-recognized system. Briefly, grade 0: normal; grade 1: rough surface or superficial zone lesions; grade 2: lesion down to the intermediate zone; grade 3: lesions down to tidemark or loss of AC; grades 4 and 5: AC loss across between 20% and 50% or 50-80% of condylar surface; grade 6: loss with subchondral bone exposure. Multiple slides (˜10), each containing five 6 μm sections sampled at 120 μm intervals spanning each entire joint were graded. Grading in each joint compartment (lateral/medial, tibia/femur) allowed for a maximum (most severe) grade to be assigned in each section, and used to produce an overall ‘average’ maximum grade in each group of mice, for the entire joint and for each compartment. In addition, a mean score was produced for each joint and for each compartment and these similarly used to produce an overall ‘average’ mean grade in each group of mice. Mean grades provide a measure of the ‘extent’ of AC lesion (representing relative volume) in each joint/compartment.

### Statistical Analysis

Statistical analyses were performed using either GraphPad Prism 6 (GraphPad Software, Inc., San Diego, CA) or “R”, version 3.1.1 (R Foundation for Statistical Computing, Vienna, Austria; http://www.r-project.org). Continuous measurements were summarised as means±SEM. One-way analysis of variance (ANOVA) was used to determine the effects of treatments. Normality in the distribution of the data was assessed using the Shapiro-Wilk test. Bonferroni post-hoc correction was carried out for whole bone measurements from 10 to 90% tibial length. This was to preserve the original inferential statistics across the 10-90% tibial length and results were interpreted cautiously across tibial length. Data are expressed as mean±SEM and values were considered statistically significant when p<0.05.

### Results

### Example 13: Hylin-Betaine and Xantho-Betaine Treatment do not Adversely Affect Mouse Health Status

We undertook formal evaluation of mouse welfare status by measuring several well-known health indicators after 18 weeks of hylin-betaine and xantho-betaine treatment (Guide for the Care and Use of Laboratory Animals, National research Council, 2010). Our data show that 18 weeks of treatment with either hylin-betaine and xantho-betaine does not alter weight, hunching, gait, activity, vocalisation and respiration scores as well as coat condition between vehicle and either of the treatment groups.

### Example 14: Hylin-Betaine but not Xantho-Betaine Treatment Significantly Reduces Gait Drop-Outs, but Neither Treatment Significantly Alters any Gait Descriptors

In vehicle group, 3 mice failed to complete the treadmill task (‘drop-out’) in 4 runs. In hylin-betaine group, no mice refused to run. In xantho-betaine group, 2 mice refused to run during the gait studies. Thus, the percentages of drop-out regarding the total number of runs were 19.0, 0 and 13.3 in groups 1, 2 and 3, respectively. In total there were data from 51 runs from 19 mice. The distribution of data by group and animal is shown in Table 2.

TABLE 2
Distribution of data by treatment group

| | 2014 Dec. 23 | 2015 Feb. 18 | 2015 Apr. 14 |
|---|---|---|---|
| Vehicle | 7 | 6 | 4 |
| Hylin-betaine | 7 | 7 | 7 |
| Xantho-betaine | 4 | 4 | 5 |

Our data show that when all descriptors of gait study were analysed no significant effect of either hylin-betaine or xantho-betaine treatment at any time point during the study were found compared with vehicle group.

### Example 15: Hylin-Betaine and Xantho-Betaine Treatment do not Significantly Alter Trabecular Microarchitecture but Significantly Enhance Cortical Bone Mass and Architecture

To explore whether hylin-betaine and xantho-betaine treatments affect trabecular and cortical organisation and mass, we performed detailed micro-computed tomographic analysis of metaphyseal trabecular bone and entire cortical bone excluding the 10% from proximal and distal tibiae.

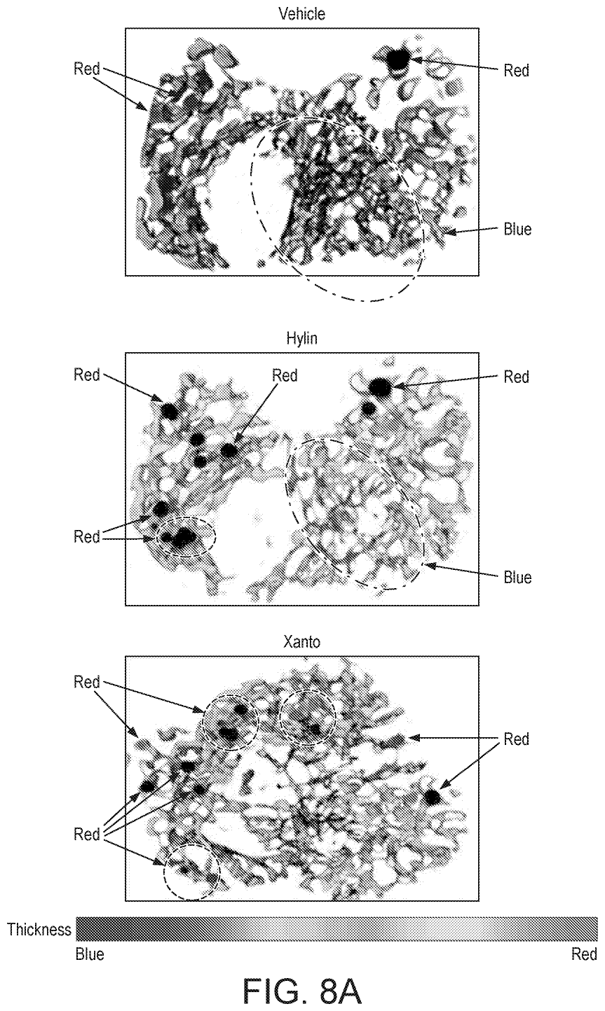

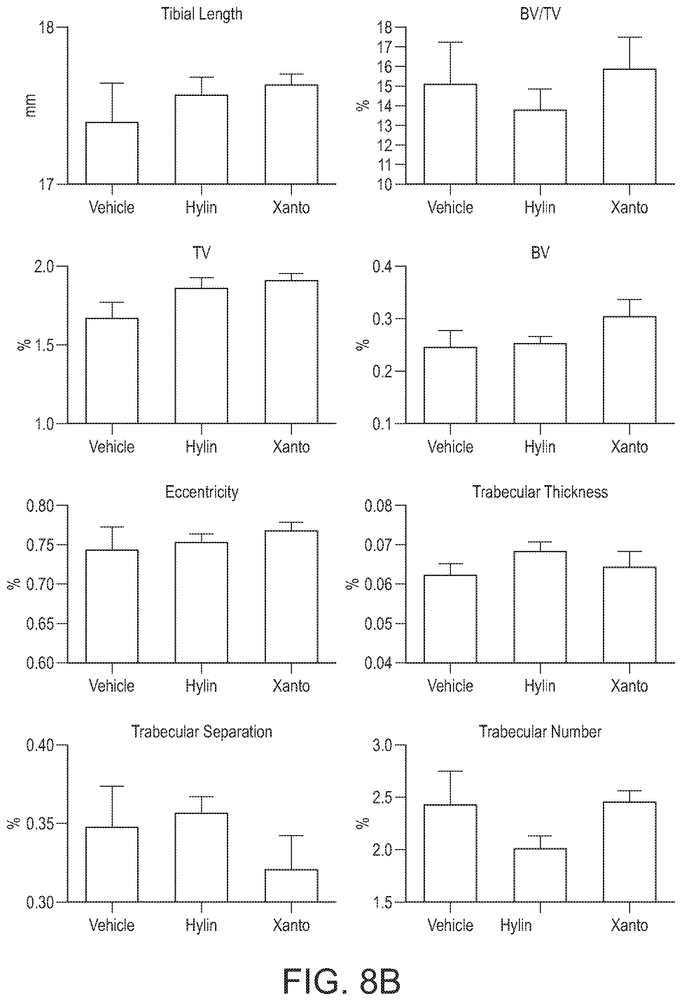

Our data show that neither hylin-betaine nor xantho-betaine treatments significantly alter tibial length, trabecular bone volume/total volume (BV/TV), trabecular total volume (TV), trabecular bone volume (BV), trabecular eccentricity, thickness, separation and number (FIGS. 8A and 8B). Together these data reveal that hylin-betaine and xantho-betaine do not alter metaphyseal trabecular architecture and mass in Str/ort model of naturally-occurring osteoarthritis.

In addition, we undertook whole-bone cortical analysis. We excluded the first and last 10% of total length, where there was significant trabecular bone volume, and removed the fibula by manual segmentation.

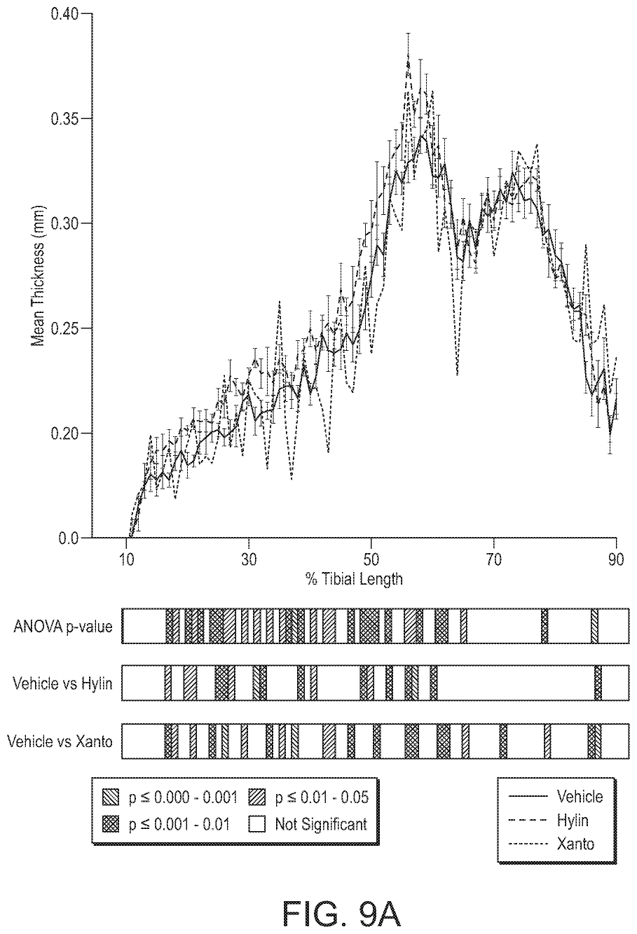

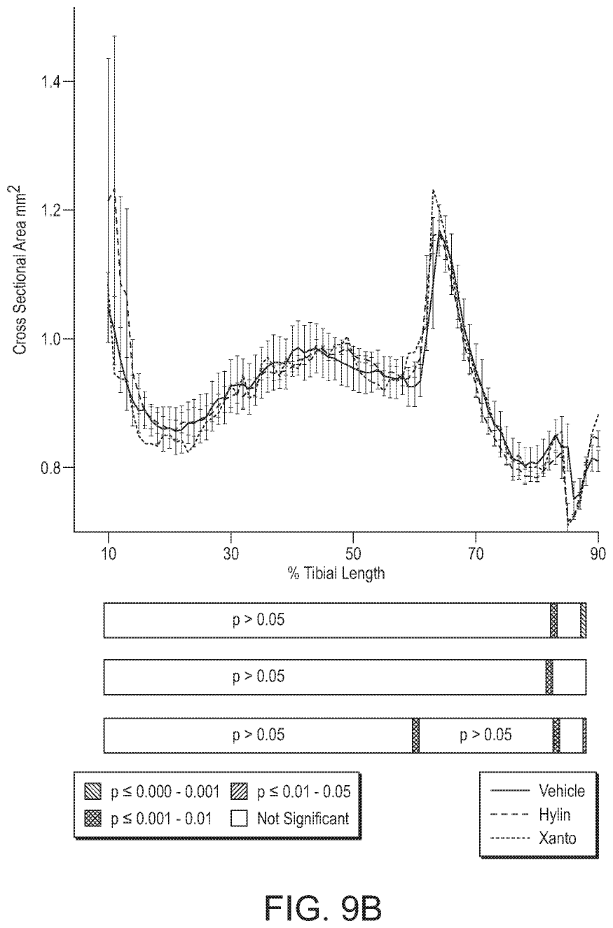

Our examination found that hylin-betaine treatment significantly enhanced mean cortical cross-sectional thickness across multiple regions from −15-60% tibial length. The effect of xantho-betaine on thickness was higher in some regions and lower in other regions compared with vehicle treated group (FIG. 9A). Hylin-betaine and xantho-betaine do not significantly (apart from one distal region in both groups as well as a region close to tibio-fibula junction) affect cross-sectional area (CSA: FIG. 9B).

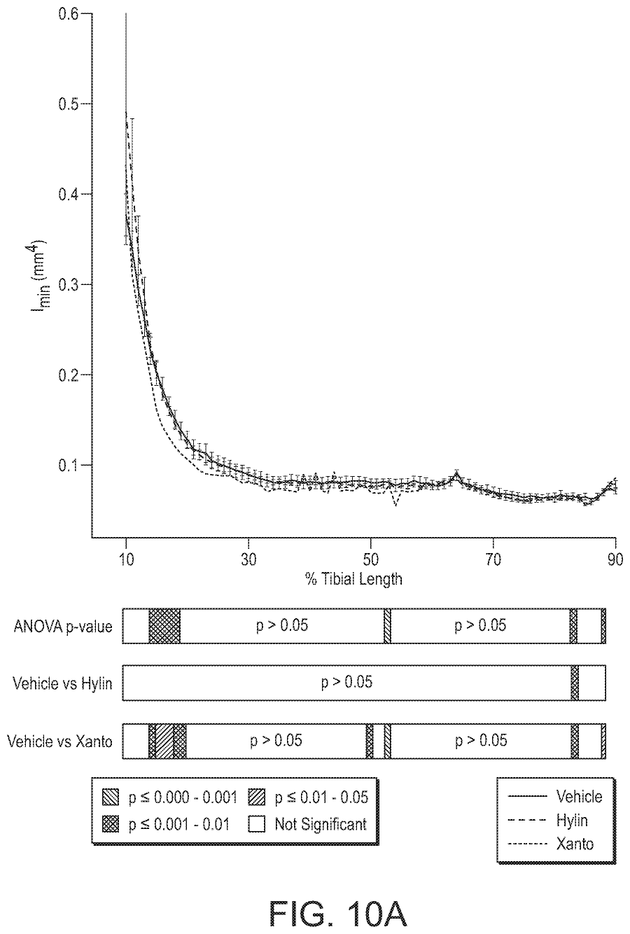

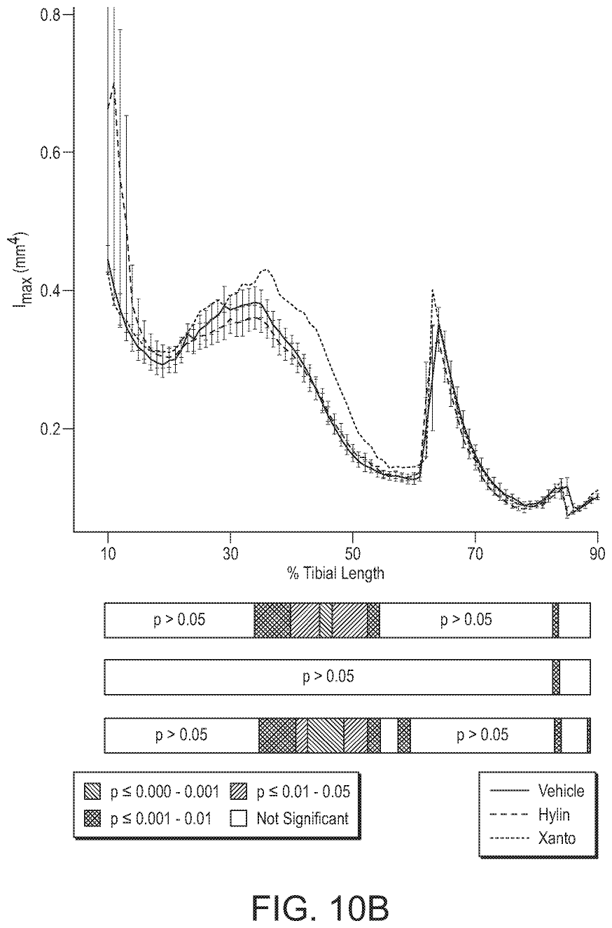

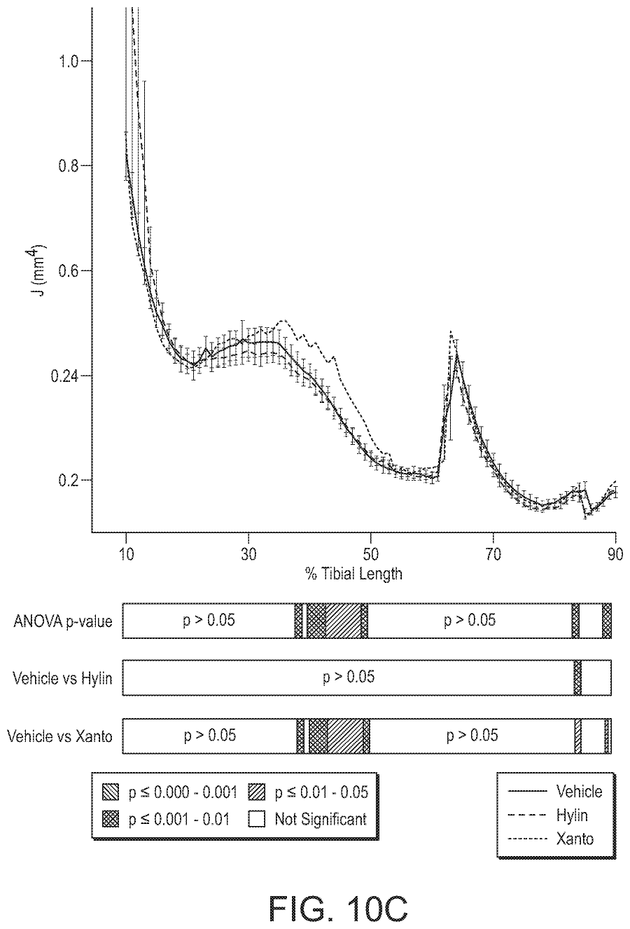

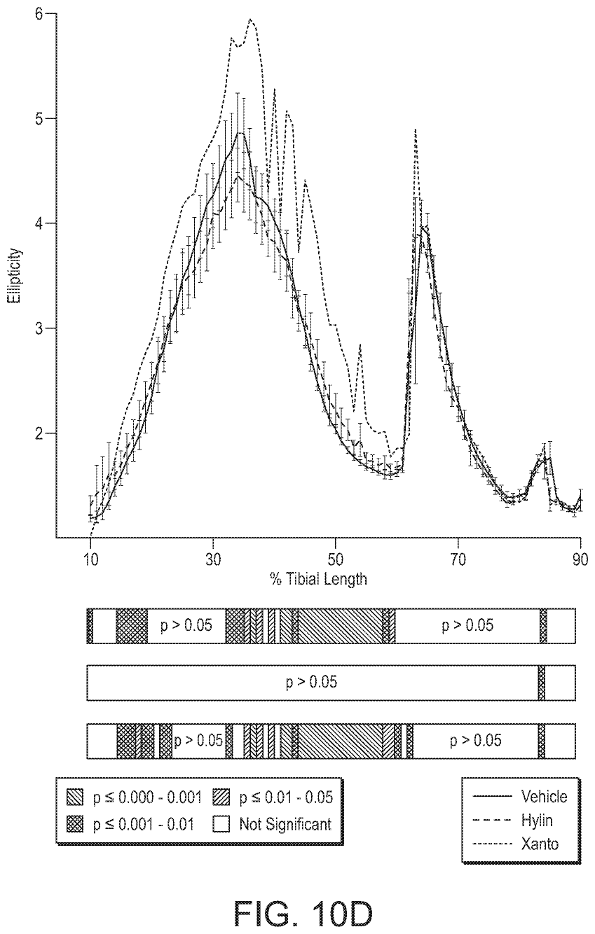

To provide an estimate of tibial resistance to bending forces, we also calculated the second moment of area around minor (Iₘᵢₙ) and major axes (Iₘₐₓ). These data show that hylin-betaine does not affect Iₘᵢₙ but xantho-betaine significantly lowers Iₘᵢₙ in one region proximally (FIG. 10A). Furthermore, hylin-betaine did not affect Iₘₐₓ but xantho-betaine significantly increased Iₘₐₓ˜35-55% of tibial length (FIG. 10B). Tibial ellipticity was also modified by xantho-betaine but not hylin-betaine treatment (FIG. 10C), with one region between ˜40-50% along the tibia showing greater ellipticity in xantho-betaine compared with vehicle treated Str/ort mice. Predicted tibial resistance to torsion is higher in xantho-betaine group at multiple regions across tibial length (FIG. 10C). Our data indicate that hylin-betaine treatment alters cortical bone mass (greater cortical thickness), whereas xantho-betaine treatment leads to changes in the organisation of cortical bone and shape which provides a significantly enhanced resistance to torsion compared with vehicle group.

### Example 16: Hylin-Betaine but not Xantho-Betaine Treatment Prevents/Improves Articular Cartilage Lesions in Str/Ort Mice

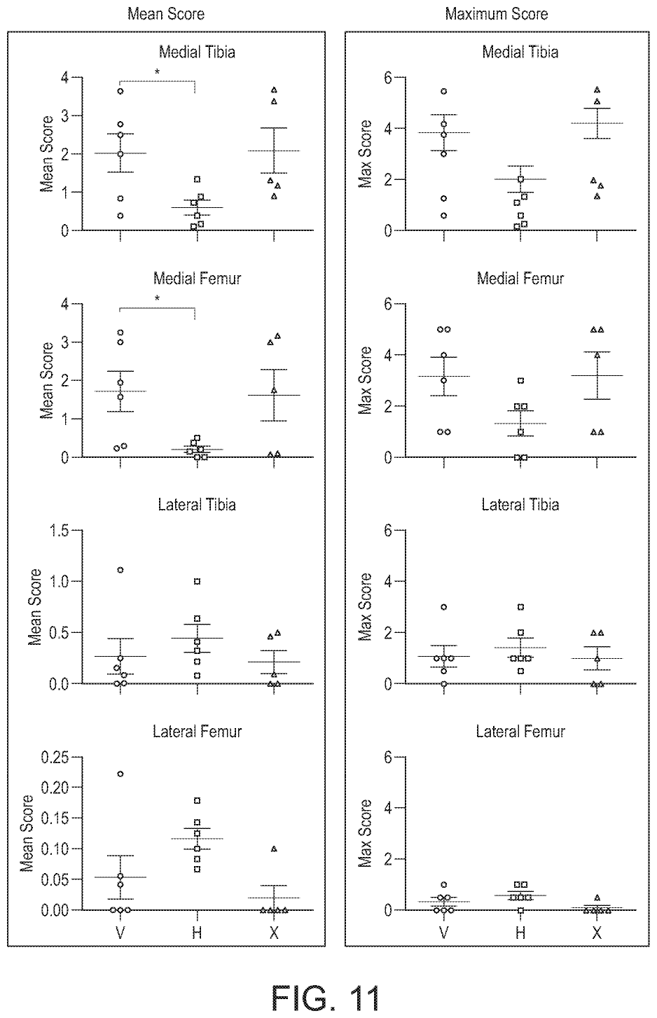

Detailed analysis of the compartment-specific development of OA in the Str/ort mice reveals a protective effect of treatment with hylin-betaine but no such protection in response to Xantho-betaine (FIG. 11). Two independent, blind scorers found that Mean OA scores were statistically significantly lower in both tibial and femoral lateral knee joint compartments (known to be where OA initially develops in this strain of mouse) in response to treatment with Hylin-betaine but not Xantho-betaine. Scores for the maximum level of OA in each mouse were not found to be statistically significant, although a trend for lower maximum score in H but not X was observed compared with vehicle treated Str/ort mice.

It is considered that xantho-betaine may be less stable than hylin-betaine (converting to isoxanthol-betaine) and that this may contribute to any differences seen between the two.

1. A compound of Formula Ia:
[hyaluronan production inhibitor]-[labile linker]-X (Ia),
or a pharmaceutically acceptable salt thereof,
wherein the hyaluronan production inhibitor fragment is of:
formula A 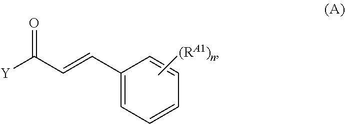
wherein Y is a group selected from the group consisting of: 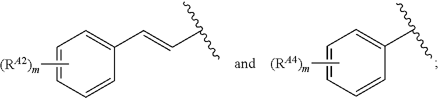
R^{A1}, R^{A2}, and R^{A4} are each independently selected from the group consisting of —OH, —OR^{A5}, halo, C₁₋₆ alkyl, C₂₋₆ alkenyl, C₂₋₆ alkynyl, which C₁₋₆ alkyl, C₂₋₆ alkenyl and C₂₋₆ alkynyl groups are optionally substituted by one or more groups selected from halo, —OH and —OMe; and one R^{A1}, R^{A2} or R^{A4} group is 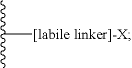
n is 0 to 5;
m is 0 to 5; and
R^{A5} is C₁₋₆ alkyl, C₂₋₆ alkenyl, C₂₋₆ alkynyl, which three groups are optionally substituted by one or more groups selected from halo, —OH and —OMe;
formula B 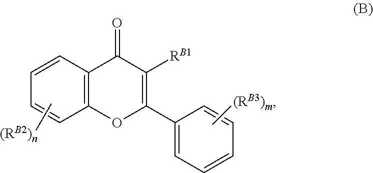
wherein R^{B1}, R^{B2}, and R^{B3} are each independently selected from the group consisting of —OH, —OR^{B4}, halo, C₁₋₆ alkyl, C₂₋₆ alkenyl, and C₂₋₆ alkynyl, which C₁₋₆ alkyl, C₂₋₆ alkenyl and C₂₋₆ alkynyl groups are optionally substituted with one or more groups selected from halo, —OH and —OMe; and one R^{B2} or R^{B3} group is 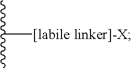
n is 0 to 4;
m is 0 to 5;
R^{B4} is C₁₋₆ alkyl, C₂₋₆ alkenyl, C₂₋₆ alkynyl, which three groups are optionally substituted by one or more groups selected from halo, —OH and —OMe;
formula C 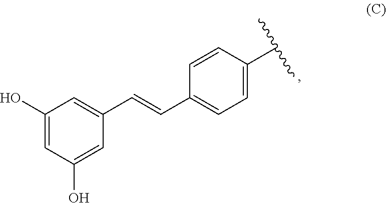 [SMILES: *c1ccc(/C=C/c2cc(O)cc(O)c2)cc1]
wherein the wavy line represents the point of attachment to the labile linker fragment;
formula D 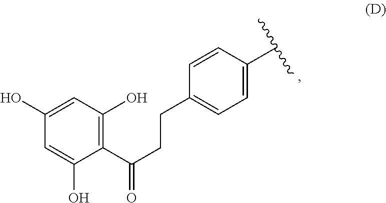 [SMILES: *c1ccc(CCC(=O)c2c(O)cc(O)cc2O)cc1]
wherein the wavy line represents the point of attachment to the labile linker fragment; or
formula E 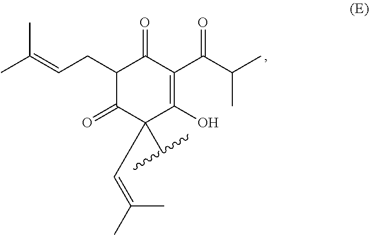 [SMILES: *C1(C=C(C)C)C(=O)C(CC=C(C)C)C(=O)C(C(=O)C(C)C)=C1O]
wherein the wavy line represents the point of attachment to the labile linker fragment;
the labile linker fragment is —OC(O)R^{L1}—;
wherein R^{L1} is selected from C₁₋₆ alkylene, C₂₋₆ alkenylene, C₂₋₆ alkynylene, arylene, or heteroarylene, which five groups are optionally substituted with one or more groups independently selected from halo, —OR^{L2} or ═O;
R^{L2} represents H or C₁₋₆ alkyl;
X is selected from the group consisting of quaternary ammonium, quaternary phosphonium, pyridinium and thiazolium salts; or
X is 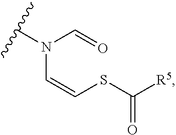
wherein R⁵ is C₁₋₆ alkyl.

2. A compound according to claim 1, wherein the hyaluronan production inhibitor fragment is of formula A or B.

3. A compound according to claim 1, wherein the compound is of formula II:

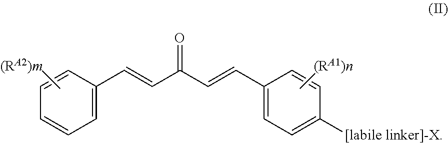

4. A compound according to claim 1, wherein the compound is of formula III:

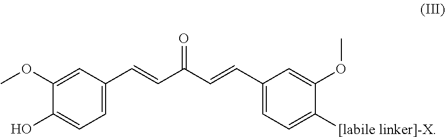

5. A compound according to claim 1, wherein the compound is:

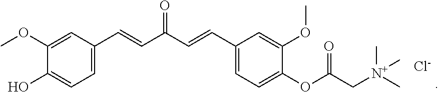

6. A compound according to claim 1, wherein the compound is of formula (VI):

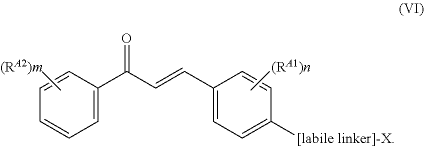

7. A compound according to claim 6, wherein the compound is of formula (VII):

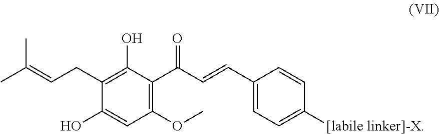

8. A compound according to claim 7, wherein the compound is

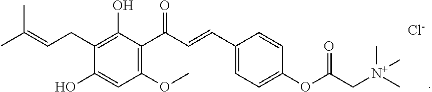 [SMILES: COc1cc(O)c(CC=C(C)C)c(O)c1C(=O)/C=C/c1ccc(OC(=O)C[N+](C)(C)C)cc1 ; [Cl-]]

9. A compound according to claim 7, wherein the compound is:

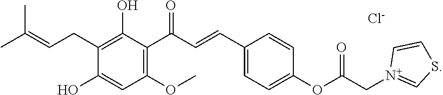 [SMILES: COc1cc(O)c(CC=C(C)C)c(O)c1C(=O)/C=C/c1ccc(OC(=O)C[n+]2ccsc2)cc1 ; [Cl-]]

10. A compound according to claim 1, wherein the compound is of formula VIII:

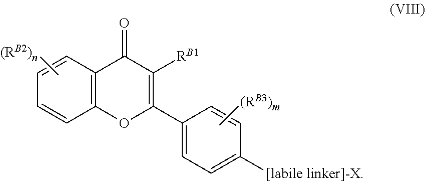

11. A compound according to claim 10, wherein the compound is of Formula IX:

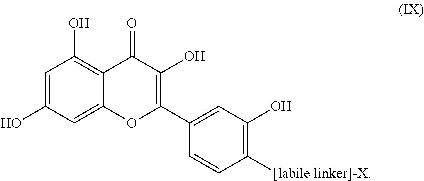

12. A compound according to claim 11, wherein the compound is:

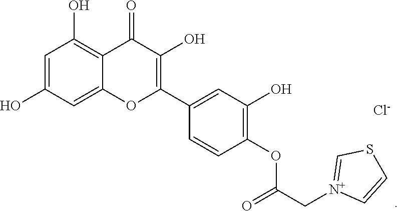 [SMILES: O=C(C[n+]1ccsc1)Oc1ccc(-c2oc3cc(O)cc(O)c3c(=O)c2O)cc1O ; [Cl-]]

13. A compound according to claim 1 wherein the hyaluronan production inhibitor fragment is of formula C, D or E.

14. A compound according to claim 1, wherein X is selected from the group consisting of: 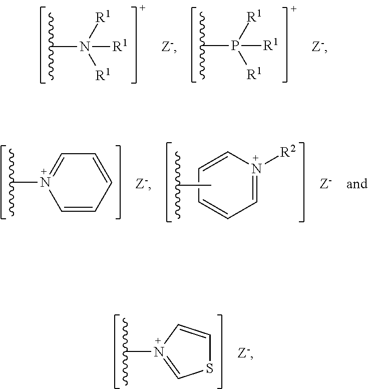
which 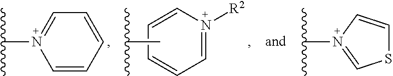
groups are optionally substituted by one or more R³ groups;
R¹ is C₁₋₆ alkyl, optionally substituted with halo or ═O, C₂₋₆ alkenyl, optionally substituted with halo or ═O, C₂₋₆ alkynyl, optionally substituted with halo or ═O;
R² is C₁₋₆ alkyl;
R³ is C₁₋₆ alkyl, C₂₋₆ alkenyl, C₂₋₆ alkynyl, aryl, heteroaryl;
Z⁻ is selected from the group consisting of Cl⁻, F⁻, Br⁻, I⁻, p-tolylsulphonate, methanesulphonate, acetate, benzoate, salicylate, or R⁴CO₂⁻;
R⁴ is C₂₋₂₈ alkyl, C₂₋₂₈ alkenyl, C₂₋₂₈ alkynyl.

15. A compound according to claim 14, wherein:
the X group is 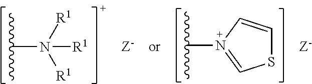
Z⁻ group is R⁴CO₂⁻, wherein R⁴ is C₄₋₂₈ alkyl or C₄₋₂₈ alkenyl.

16. (canceled)

17. A compound according to claim 1, wherein R^{A1}, R^{A2}, R^{A4} and R^{B1} to R^{B3} are selected from the group consisting of OH, OMe, C₂₋₆ alkenyl.

18. A pharmaceutical composition comprising a compound according to claim 1, including a pharmaceutically acceptable salt thereof, and optionally one or more pharmaceutically-acceptable excipient.

19.-33. (canceled)

34. A method of treating or preventing a disease characterised by hyaluronan overproduction comprising administering to a patient in need thereof a therapeutically effective amount of a compound of formula Ia, or a pharmaceutically acceptable salt thereof, as defined in claim 1.

35. A method of treating or preventing a rheumatoid disease comprising administering to a patient in need thereof a therapeutically effective amount of a compound of formula Ia, or a pharmaceutically acceptable salt thereof, as defined in claim 1.

36. A method of treatment according to claim 35, wherein the rheumatoid disease is arthritis, optionally osteoarthritis.

37. A method of treatment according to claim 37, wherein the diseased characterised by hyaluronan overproduction is cancer, optionally wherein the cancer is sarcoma, or chondrosarcoma.
